SEQUENCE LISTING (31 sequences)

SEQ ID NO: 1 - DNA, 22 bp, artificial
  Synthetic oligonucleotide
gattgatctatatgatgtacgc

SEQ ID NO: 2 - DNA, 19 bp, Artificial
  Synthetic oligonucleotide
ccccaagcaaagtagttgc

SEQ ID NO: 3 - DNA, 348 bp, Mus musculus
gattgatctatatgatgtacgcagaagaccatggtatgatgttttgactacctctaaagatgcgatcaataaatgggggttctatcatttgttaatacattgaggggaaacttccccttagtgggatgggtactctcttatatagaatgctgtgatactttcaaaatacaaatacaattaatgacaaatgaaaatgatgtgtttgagaaggaaatctctgtgaatactgataaaatgattttcagactcaattcaaatggattgctgtaagtcttctgtccagttttagctttcagtctcctttagctggcagggcttttcttcaaagagcaactactttgcttgggg

SEQ ID NO: 4 - DNA, 16375 bp, Mus musculus
tgctcctcaagtgctgggattaaaggcccatttgaagacattgctgactctaggaaatttgctattataataagagtatgcaggcagccatacttggtggtacaaatgaacacacactcacaaacccacacatacatacagagagagagagagagagagagacagagacagagacagagacagagacagagagagaaagagagtgagaaggggggagaaagcctagagcttcagcaaaaagagaaaggtaaatgagtaatgggggattaagcaaagaactaagctttattatagagatgtatgcaagtgtcaaagaataaaggataggagaaacaagacttctctagtctctcagttgtctgtctacacctgaggcagaggttgctagctgctcacaaattctgtcatccatttataagtgtcctttgcaaatgatgacttgactgaggacttaatttttttaaatttaattttattagtaattcattttttacactccatattccattccatgcccctccccatccaacctctgactgctccacatcccacacctcctccccacctcaccctatctccacgtgaatgctcccacccccaccccacctgatctctaaactccctgggtccttcagtttcttgagggttaggtgcatcatctctgaatggacacagacccgtaagtcctctgctgtatgtgtgttgggggcttcatatcaactagtgtatgctgtctatttggtggtccagtgtttgaaagatcttgtggatccagattaattaagactgatggtcctcctacagaatcacccttttcctcagcttctttcagctttacctaaggcctgacactattactgaggctatggaatgctcacaaaaagggacctatcatgactgccctcagaaagacccaacaaggaactgatagagtcagatacagtcatttgcacccaaccaaggactgaagcagccgaggacttaattttttaaagtccctctgtatctgtggtacccagaggcagtccacactaagaaccatgaatgaaagaagtctatcacttcaagactcacactgcaatgacttggttgtacctcatcctcaatttttttctagctctagctcttgggagaccctgggactgaaagactgtcagaaacaataactctggctgtcctagatctctgcataattgcttggaacatttttagaatttatctgtttgtctctctatctactgtctgtttgtctgtatgtctacctatctatgtacctagctatatgtgcattgctgtttgttttgagaccattctcaatatggagccttggctggcctagaaaccaaattaacctcaaactcaccagtctctgctaggcacatgctggcattcaaacccacctgcctctgctaggcacatgctgggattccaggtgtgtatcaccacactcccccttgaagtaaaatttatgatacacatgaatagtgatgcctaaactggaaatcttagatgacaaataacagtactgtgataagacatgggtatttgtgatcatactttgaggcatttcctgttatcaaaactaatacagattagtcttccttttttttttttttttgagtttttccaagaaaaatcaaaatctgaagaagtcagccattttcctttctaaatcctctgggacttcagtctgagtataatatccccactcacttaagaagaccaaccctccctgcccccacacccgcgctggaactgtgaagcaaactaggagctacggagattctattctgaaatagaactctggatagtagtatggaatctattctggatagcaactgaaagttctgctaggtatattagacaagttgagtcaactaggctacttcctttaaaagctagctagagtttattgtgttggaatgtaacatgtatctaatactctgcagtacatgactgggccaagctctgtatcagtggcattctgtctgccagaaaatatccgcagtgactattcagacatttttggagtctataaaatgtactgttttaattttagtaagattaacaatgtgaagttcctagagttaaaaaaaaagcaaaacaaaacaaagaaacaaacaaaaaacaactttgcacataaccaagtagaagtgcaattatctgtcacttgaagtggtgtttctactaatagacatggttctcaacaaatgaaatgaaaaatggatactttttatcattaggcaacattcataaaaaagggggcatggaagggaactgcaaccctataggtggaacaacattatgaactaaccagtaccccggagctcttgactctagctgcatatgtatcaaaagatggcctagtcggccatcactgcaaagagaggcccattggacacgcaaactttatatgccccagtacaggggaacgccagggccaagaagtgggagtgggtgggtaggggaggggggggagggtatgggggactcttgggatagcattggagatgtaaatgaggaaaatacctaataaaaaatatatgtaaaaaagggggcatggaaatgcgcatgcctccttaagattgatttccagaacatgatgtttaagttctgaatttatgtacaggtcctactccctgaccaaggggttggagagctgctggagtacactctacatatcatgtaaagtggagtaatcctgcagtggacttagtagaagacatattgatagcttgtttcttttttggtctggggaaacatttccataaatgatcattatcgctgtcaatgaaatgatccattcagaaatgagattaccacactatgctgtttctaaaaccccctcttcagccatccttaagtcttaatggaacacaaaaggttcatttaaaatgaaaagtggtttccccttttccaagatttgagaatctctcatttcctttccagaagtctagttttcctgtgaatggttataaagcattatttgaagggactgcagaacatagtcattttttctttgctattttattttccataattttgaacttgaataaatagcataccacttaaataccatgatgagattttatacactaagtagtaatatcacagaagaatttatattgtttaatttaaccataatataaatactctaacttattattactgtgttttacttcaggaaaacttatatgagattaacagttttaaatatatgccgtctaagattagcaatgttaataaaatttaatgtccttgtccttaatgtaaattatagcattttaattttcttgtgtctttaaagaaatgtataaattttatggagcatattaattcctattagtagatttttatttagcaatcttagtgaaaacaaaagtttcatactaatgttctctataaagaatgaaggacattgctcagggagagagagttaaaatatatgcaaacatgaagacttaccaaagtgcagatttctcaacttcatgtgcacattttgtaggtgtgttgactcagaaggtagactgtatcctcagaacaagctggatgcaagagtagccatattaatgagctctgcatttgattgtgactgctgctgtagtgaagcaggtggaggagccatgcagaatgatctgaacttcagcctaggacaaccatgtgtactgacatgtgcgcagccatcacatgcaaggcaggaaaataatcaacaaacatcacaatggggagagtgaaaacatttatacttttaaaaatccaatctaattcaatcaattatgagtcattgttgagatcttttaactcatggctgttggctagacattggctatggaatgtaatcacagtcttatcagctctgtgcatttctgttagctttggaaaaatcaaaagggtcaagagagaaagtaaaccatttggaccagggaaattttattgttaagaaatagttattcactataacttaggagatggctcagagagtaagacgactctctgtacaaccataagaaccggagttcagcctcccagcataaaaatctgggtgtagttgtaaccagttatcccagaattgtgggaggggctgaattaaaaggcttgctggctcccagtaaatagtgctccaatagtagattcaatgtactactgtggtctgcaggagtgctaagatgtatacctacacacacatttgagttttcaccaccaatatataccacaaatcaacacatatcataatggggaaaagaaaaaatatactgtaaaaaatccaatctaactcaatcaattatcagagtatcagagtcattgttgagaattaagaaacctaaggctaaggacatggcttgggaagagaacattaaaatgtatgcaaacatgaagacttgacagggtgcagatccctagacttcatgtgaacattttgtaggtgtgttggctcagaaggtggagaccagtatcctcaggatgagaagttttattagaattaaagatttccaaaaaaaaaaaaaaaaaaaagaaagcacaaataccatgtatatgacaaggtagtctaatctatagctacctatgagaatgaaataaagtcataaaggagatattaatggcccaatactctgacctaagatctgatcctcttctagagacatggatatattcttaaaaggcaggtaagtcactaggaggccattttggtggaatttgctagaaaatgactctttggtgtttctaatgttaaccagaaattgtcgtctacaaaaaatatataaataaaaataaaacaataaaaatcgtgagagaggtcctatagatgagaactcatgtcagtaggctattgttggccattgggatcgtagtggagtttcagaatcttccagaacaaggagaaatagggactgtataaaacaatggtggctaagtctaaagaacccgtatgtatggcagcagacagacaccagataatctgttcataggcagagcccatgagctaacaagtgacataccttaggaaatatacctttctcattcagtgtcttgcatcatctgtacataatatatggaactgtgctaattactaaaaggaaagtattgaaaatccccagatattcaatcaaacaaaccaaccaaagtgaaacatttcagttgagaggtaatgaatacatatatagaggcattgtattaaaatttacatataatttctttaaattattttattattattgagatattaactgatattaagagtaattgttgaaagggaatcaatatatcatttcacatacttgttgcttaaatctaagactattccaaggaaaagaatgatatttatatcattgacagcacggatgaaaactgaactcattttaaagaaataagtttaatggatgttatattagctatgtcagtaattggttctttggggagaactatatttaaacttacagtttatgcaaatgtgattagtcatttgaaatgaataagtcatttcataatatagctatgcatataaaatttcctagtcaagaagggcttctgcggttgtatatgtattttgacatgctttaattggcccaattatcttagttggataagcatggctcttgcaaagaatctccataattttcatgatttctcaataaaaaaggggggggggtagatcatagtgcatttttcatctgagtcagggattttctttagtcacatgtataaacatgcttgatgtaagggaattctgtggcataaaattgtcatgcagcccatgggtgttgacaagtcttcattaggtccagcctgtggatgaaagtaatgagttccagagcactttaatctaagtaaatgtagtcttaattagaataagtatgaatcaattacctttcaaaatcaatataacaactcaagaaggtttttttttgtttgtttaactttctaatttcagaaaaatgtaaaggtttcaaactaatagattattgactttctgtttaaattattttacatcattcaaataaatttgcaaaaaccaaatactttttagtatatgtgctgccgaagcgagcacaaaaaccaaatactttttctgttatgttttaaaaatgatatctatctattaaatgcattcgatgtatttctatctcagcacactaccctttgcctttccaagtctttcaagacctctttccctttagggtcctcctttccacttgatgtgcacttgggtccaattaaagatgtccttatgtgtgtgtatatagcaatctatgtgagcatgggcagtcttccaagcaagttactacatctctgaagaaagccaacccaactcaccttcccctaataaccctcccctgggccctcatgtgcatctctctgctccgggctagactgtggactgacttgatgatgtgccggtctcagagctgtagatgtcatgaggtcctgagtgtaacacttgtggaagacgctgcttgacagcagtcctccccattccctagctcttacagtcagtgcaccacctcttcagtcatccttgagtgtgagagggaggcagtgtgacaaagatagcgactttggggcttgaaacttcaccatcatttattccctaaactttgactagctgtgggtctctatattaatgtctgtccactataaaaggaacttctctaatgaggagtgaaggctgcaccaagccaccaagaatctgtattgacatgagtttaatagaaaaagagaattaaagttaactttaattgtagcttaaccttttatgtataattttaatctgaaactgaacatccaatttaaagtggtccattggaaaagatagttgataaattatttcaataatatacattatttattaagcacgtgactatgaagttcacaaccatgctgctctgtgatagaagctaaagacaaaaaatatttttatgggagaaaatatcactgtttagtcttggctaacctggaattccctataggccaatctgtctttggactcatagagcctctgctgtccaagtgctgtaatcaagggcatgtgccatcatacctgggaaaaacagtcctgcatatagtcttgtatatacagtttgcattgaataaaaacagagtgggcaattaattgaaagcaccacctttactagctgcagcaacattttctgtcaaagaaaagcatctcaaccaaacccctgtgtctggagcttgttcggacaatatagggagagggaattggcttgtgttgggatttcttttgtactgctgtgttactttgcaattgtaaaaaaaaagaaatgacataaaggtggtgttttatatttgaatattcatataggtaaatggaatttggtttatcttttattttgttattttaagttgcctattagttttgtaaatagatcttattgttagaggtggtggtatttctcatgccatggcatgcacatggaagtcagaggacttgccaatactctcctcctgccaggatctccttctgtgtgattagagatggaattcatcttttcggtcttggtctgagcacctgaactcactaaggcatcacctaagcccagttatacttagtactttggcttagtttctctgatatactttgtttttcagcttctccatgggtggataatagtagaactccaaacaagattgatctatatgatgtacgcagaagaccatggtatgatgttttgactacctctaaagatgcgatcaataaatgggggttctatcatttgttaatacattgaggggaaacttccccttagtgggatgggtactctcttatatagaatgctgtgatactttcaaaatacaaatacaattaatgacaaatgaaaatgatgtgtttgagaaggaaatctctgtgaatactgataaaatgattttcagactcaattcaaatggattgctgtaagtcttctgtccagttttagctttcagtctcctttagctggcagggcttttcttcaaagagcaactactttgcttggggaaaagccctcacatagagatggcatggagtcacagacagcttgcctctggcagtgaactaatgggaactggccctaagctaacagtgctgcctagatacttccataaagtttaggttttattatttctccccaaagtaattttatttgtttgacactattaacgtaattcatcatcctaattcaaaacacaagaaaatatgaacaatatatgtttgaactctctgggagacaactatgaaacttcatgtatttttattattgattggattttgagtgtataaaagttatacttaaattgtaaatatactatttttaatgagttcacatatacaacatggtaccttatactctgttcttttcatattaatttttgagtatgtagtaatgaaaaggaatacttaaaatccctttaataaaggctatctgttcacttatttaagaaagtccttattgtgtttgtgcattgcttccaacttctggtattttcacacaaattctgtgcagcatagtggtggttgaaagaacatttagttatggctttcaagtgtcctgttgagtattcaataggtatttgcatgaacacaaccttaagtaatagttaccaatgtattagtgacttttaagtgtccttctgaatagccaatagcaatatacatgaacaaaaccttgaatactattaataatatttctctagtctattatttaatgaaagcaaatactatgttataacatagttgtacttaggaaacggtcattttatgggaactgtaggagcgaggtgtcactcatctgaagcagacactttgctaatcttttcatctgccactgagcagcacaccaggacaatgtgttaagatgctagacatgagctgggttgtcttgcatgggcaaacagtggagctttgcctattggtctgaatacagagcatggagtgggttgcaggcaaggcgctggagtgctcattctacaaataatcccaagtagtagcagctgtgctccgaaatgctgagaatgaattggcttgctttctgtctgttgctgggtcacttgagtcagatgcagcccttcccactgtggaaggcagatgcttaatcccttcaatttgttttgttcccctccagtctctggctaagcaatatgtgtcttctctttgttccttttctttctttttcttttcttttaaattactttgctgctatcaaatttagctctctacaagcacttaacttcctgcctaaatctcccgaagtgtgtatttcaaatacagagaatcagagatgtggttagagtttggaatttacatttaaactgtttctctggtgattcttggatgccataaatcttttagagtttctgcttcatatttctggtaatagtaatatccattacctggtttcagtttttaacttacctgttatatttccttttaatttgctttatcttaaggctttttgactacttatctctctgtgtttatttattttttgtatttaaaaatgtcataatttgttgttcactacaactgaaatgtcagcaatcagaatttaggggccgggcattgtggcatacgtctttaatcccagcacttgggaggcagaggcaggcgaatttctgagttcaaggccagcctggtctacagagtgagttccaggacagccagggttatacagagaaaccctgtctcgaaaaaacataaataaataaataaataaataatcggaatttaggattgttccttcttcagaagtaaataatcgtttggaaagtaattttagtcactgcttacattaaatttcatctttatttttttcataaatccctagaaactatgcattcaaaagccataatgtattaatatctctaatgggatcttaaatactcttttcaaaaaaacatttcagagtatgtttaaaagagcattgtttactatggtgtaatagaatgggactttgttttaatcatatcatatttcagaaatgtataaatatgaacacagcaaatgtcaatgttatacatagaggaaaaggcaatgttttgctggttctgtttcaggtacatccagggagctgcatcccccaaggacatgctcattctggtgaacgtgtgagtagtgcatgagacatttgtaggtaataataggttgttgggaattagaagtgtatgaaagttgggataacaaagaatcaatataatggttgtatatctagaaaaacaagccaaacttcgatattaacaattgattgttgacttggctcaatcagtaaagtgcttgttgcacaagaggaaggtctgagtgcatagctctggcttttacacgaaaacaattatataataagggagcagtggttgtgcctgtaatgccaattgtgaatagtaggagagaagaagtgccccagggctcactgattagacagtctaagggatcagtctgttctgggattactgtgagaccccagtattaaagaatatggtagagagccacagaagatacctggcattgacccctgacatccacacacatacactcacccacccattccacaccccacatttatgcaaatgggcacacgaagacaataacaaaaccagaaagatcattctattgaaaatgtaaataatgtatctgtatcaaactgggaaattatactattctaaaagctaatgagatcacttttgttctttatgatcgtgtttgataaatgacaggtgaatgacacacggacccatttgtctgtttgtaggagtggaagtgtgagcggattgactctgaaactcatccgaacatctgtctccgagatgttagaaaccctctctgatgatgatttcgtgaatgtagcttcagtgagtatggatggctgctccttctggaatcccgcctgcctgtctccctacatcattacatactgggttgtgttttccttcgatgggctgaggcattgtcagagtgctataagctgtcttactgatctagcctgtattgtcctttctgagatgcttagcattaccaccactgaagtaatgaatactgaatgaaaagcattccgtcccttccctcaaaggagcccacatgggtactgtgagcccttccacacacaggcacctttgggacatcttatgaagtagcatgattgaactgttaattgcttgtagctggttatactcatttgtgggctggacagctgaccttattgcactgagtttttcctaacagaagaaagtaaaggggaaagtgaataagctgggaattatcttagaccttttcctagctaggaagtactggccttgcggcaaatgggccaagatgtgccatgtctcatcttgatctatgtaatttagggacccaatttcatggccattttctagtgcaggaaatttcatatgtgaagtggttcatttatggagatgtggctgaaggagcagtagattttggtggtgttgatttttattgaacaaaggagaatagatctgcttctaaaaatcactgggtagggtgaagttaaagtctacaactgattgtctcactttcctggaattgggggaaacagaaatctccataatcctaattcttctaatgatgatggaatggtgatgctaagatggagaggatttatctctaaaatgaaatcactaagatagtcctgaatgctggtatggataaagagtatagaacccactatggataatgtaagacttcctggatagtgtgtcacgatgtataattctcaaggagaataattttaaaatattctatagttcaaataaattcagatggaaccttgtcaaactcttgttgagacattatggcaagatgaccacttcaattaagatataggaaagagcttcatttgtatataaatgtatcattttaaaaaaaaaactttggaatagcttaaaatgttatccttggcagaatgagagtggctcaaatatatttagttcagatttgtcatatataataaaaattatttaagacaagaaaataattaatactattaatgttttctttggcttctttcaaaaactgaacgtcagtctgagttgaagtagctcctttggaaatcacagggaatagaactggttcaacagtgagaggtgaagtgtgcaaagggagaatctaattggatattcttcatttaatttaaggaaaccaagtttgttgagaaattctagaaacctccaggaatagtcttttttttagagttgctaagctacaaaccctaaagacccagtgtgttaggtaccagcctcccttatgtgtagatgaacagaatatggccacgagcaggtattctgaactgggaaaattatggattctgaatgttgaaagctgatcagtgcataccctagtcaatgaatcagccataaaaattgacattatcacataataataataataataataataataataataataataataaacaataaacaactgtgatgatgatgatgatacacagaacattttataagagggtggtgagcttaaagttagtgtctgaggggaatccatgcagctgttctatagacaagtaagtggcacaaagaggtaataatgaaaactggtacagactgacttcaacctggatcagactttgaagtgtctaatgccaggtggtttttaatcccaatatatttaagcccagtataatttggaaaaaaaacgtgttctgctgtaatacagtcccaagtacccaaggaagggtaaactctactcaaggtacatcatgaaactctaatcagagtatgtgtcatgttgtcttccaagaaaatgggctctagagataggctacctgtactagtttaagggcctaacggtggcctggttttagtcacacagatagaatacaggtcatctgggccttgttgtgcaaatttgatatgacctgacccaccaataatgcagatcaggctattaatcagttccaactcttagacttctgaattgaagaacaggtttttcatctttcccattggatagaatcccattcacttaatattcctgatttctctggatatctgcgggcacagggatctttatactatgcctccaagaagtgtcaaatttaatagtattttgtagtctattcattttttttttgttcctctgatactcattccaatttccctatactcttgcccaaccttgcatcagcatcagtagcttggttggcacattgccttacatattttaaacaggggactttgaattcaatgtccattcaaatagtaggtctatgattgtctattcatagacattacaatagatgaaagcattgaatgaataccataattccatgtacagtcttctctgcctctattatattgcagcaaatttaatgacaaactattttaaaatcttggtacattggaattaggtccccaaatatccagagagcaaatacacatttaggtttagcaaattcttaatgaccgccataaatattgttctcatttccaccacatgcacatgaaatgcagaatattcctccatactccatagagacaggaagtaggcagttcttgagtgatactctgggttatagtgacaggagataagcttcctcacatttggttcccacatcaatgctttctattttctgaatgctaaaccagtaggtgagtgtgaaggcattgctgatgtttgtcatgcaaatatgccatggcaaattccaagcaacttgcaaaaggtcaactggccataaacaacgcctgaaaatctaaagattgcttttcatgaattaatataatcctgacacatgcatagagcagcatgcccagtcagattgtcttcttcatgctggagccttagcacatagttaagggtaaatagtttattcaagccagtgtctagggagatagcataagttaggccctgtaaattctttggacaagtgtctgttatcgagcttacttttcaaactagtttgcttcatttgagtgtttaagtagattcccatgttaaggcaagcagtatgtgaactctgaaggttaataatactttctgaggtgctatggtgaagtacctgggactggatttatgtcaatataagagacacacatttactcctttcctaatgagtcaacatctaacatttttttaaaaagtaagttgatgctcatttccctgagaggtattattgtatggaccctgtactgaattggtgctaatgagctggccatagctgtatccatgtgcgcagtagctatatcgaaacagatgcaaaagacctgcactcttcattctaagtatatactttctccctctctcatttcttggctacactcactgggtcatttaataatcattgtagttatctgagacaggtaggttccagaggatgaatcattttacccccacccccactctctagcaattcgactggagaagtacaatctctaggaaacatggatgtgacacacagttgtacacaattcattcattttgccgtagcttatcctcagcttcttcttcaactctgcctaagagaggttttccagtatttctccttcctgggcaaactattcaagaggcttccacctgtatagaaagacatgtatttccttattccaggagatatctggttctcgatataaaattacagtgaagtgcattttgcctcaaggcctctcaaaatccatttcccgtgtgtacttctggcttctgctgcttcatattcaacatacctgctcattctttgttgaatattctgttttcttgttttcctggcacggactgtactattgcaggtggtgtctattgaaatttggcatttttgttgcttagagccatgagagaggatgggatgaaataataaagagagcaaatgtcgttttttattgtctctctgagtggagatgccactgcactgtaatagaagggaaccatgtatatctatacaaacagatatggtcaaccgagggcacagtatcttcaagggcaccacacatccacctccttgtgtatcatcctggcatttggatagatgctgtggcagcttaacacctttctacttcacattctcaatataaatccctttcttctaggacatgttgaccacagtaagaaaggggaattggtccataaaacttttccagctttcttggtctacatttggctggaccaaggatatcgctttcttcagtggaatttaaagacctctcagcgcttagctgtacaattattattgtcttcacatttctcagtgatctactatgcctgctccatttacataatgctcagattactataactgaatctgtagtgtagtgatgccaaatgcctgagcagtcttcttggctccagttatggacagtacactccttaaccaaggctctttctttctattgccatgagtctagtaaattggtctcttcaggaatatcaacctgagcatctatttaccaagagtggtatcaaataagattagtttccctagaaacagttactgagataaagatttaaattcaaatagttgatatggaaagttactctagaaaaatctgattgtgaaattcagaaattaaagaaggaaactagtgaataatgcattgttcagtagcacaaccctgtgagagttgagctcattcttctgcccatcaggaaatagtatacaaaggtgcatattttcaaaggggagatatgctcttatgtgaaatgactaacccatgtaacatcagttccctttctttcacagcatgctgtcttcagtgcagargttggagtgtagtcttggtcagattcaggttatgttggcatgtacagaacgtatggccaccagtaacatctgtccaagtaagacagtgaacaatgctattgccaaatcctaaaagaattttctgatgacatcacctttgaataggtattatgcattcaacattgggccaccagttaacagcccttttcctactggtagaacacagctggagtcaaaactgagattcacaatgcctaataaaaattgtaatcattgttttgctgaagccattccaactttagatttgtacttctactattgctgtttttgagaagtgtctcagtgatttacctgcccaagtctaccacacctggctttaactatatattttttggaaagtaaacattttaaatgacttcttttttattttttttaattaatattttttattacatattttcctcaattacatttccaatgctatcccaaaagtcctccataccctccccccccacttccctacccactcattcccctttttttttttttttggccctggcattcccctgtactggggcatatacagtttgcatgtccaatgggcctctctttccagtgatggccgactaggccatcttttgatacatatggagctagagtcaagagctccggggtactggatagtt

SEQ ID NO: 5 - DNA, 8791 bp, Mus musculus
gatcttttaactcatggctgttggctagacattggctatggaatgtaatcacagtcttatcagctctgtgcatttctgttagctttggaaaaatcaaaagggtcaagagagaaagtaaaccatttggaccagggaaattttattgttaagaaatagttattcactataacttaggagatggctcagagagtaagacgactctctgtacaaccataagaaccggagttcagcctcccagcataaaaatctgggtgtagttgtaaccagttatcccagaattgtgggaggggctgaattaaaaggcttgctggctcccagtaaatagtgctccaatagtagattcaatgtactactgtggtctgcaggagtgctaagatgtatacctacacacacatttgagttttcaccaccaatatataccacaaatcaacacatatcataatggggaaaagaaaaaatatactgtaaaaaatccaatctaactcaatcaattatcagagtatcagagtcattgttgagaattaagaaacctaaggctaaggacatggcttgggaagagaacattaaaatgtatgcaaacatgaagacttgacagggtgcagatccctagacttcatgtgaacattttgtaggtgtgttggctcagaaggtggagaccagtatcctcaggatgagaagttttattagaattaaagatttccaaaaaaaaaaaaaaaaaaaagaaagcacaaataccatgtatatgacaaggtagtctaatctatagctacctatgagaatgaaataaagtcataaaggagatattaatggcccaatactctgacctaagatctgatcctcttctagagacatggatatattcttaaaaggcaggtaagtcactaggaggccattttggtggaatttgctagaaaatgactctttggtgtttctaatgttaaccagaaattgtcgtctacaaaaaatatataaataaaaataaaacaataaaaatcgtgagagaggtcctatagatgagaactcatgtcagtaggctattgttggccattgggatcgtagtggagtttcagaatcttccagaacaaggagaaatagggactgtataaaacaatggtggctaagtctaaagaacccgtatgtatggcagcagacagacaccagataatctgttcataggcagagcccatgagctaacaagtgacataccttaggaaatatacctttctcattcagtgtcttgcatcatctgtacataatatatggaactgtgctaattactaaaaggaaagtattgaaaatccccagatattcaatcaaacaaaccaaccaaagtgaaacatttcagttgagaggtaatgaatacatatatagaggcattgtattaaaatttacatataatttctttaaattattttattattattgagatattaactgatattaagagtaattgttgaaagggaatcaatatatcatttcacatacttgttgcttaaatctaagactattccaaggaaaagaatgatatttatatcattgacagcacggatgaaaactgaactcattttaaagaaataagtttaatggatgttatattagctatgtcagtaattggttctttggggagaactatatttaaacttacagtttatgcaaatgtgattagtcatttgaaatgaataagtcatttcataatatagctatgcatataaaatttcctagtcaagaagggcttctgcggttgtatatgtattttgacatgctttaattggcccaattatcttagttggataagcatggctcttgcaaagaatctccataattttcatgatttctcaataaaaaaggggggggggtagatcatagtgcatttttcatctgagtcagggattttctttagtcacatgtataaacatgcttgatgtaagggaattctgtggcataaaattgtcatgcagcccatgggtgttgacaagtcttcattaggtccagcctgtggatgaaagtaatgagttccagagcactttaatctaagtaaatgtagtcttaattagaataagtatgaatcaattacctttcaaaatcaatataacaactcaagaaggtttttttttgtttgtttaactttctaatttcagaaaaatgtaaaggtttcaaactaatagattattgactttctgtttaaattattttacatcattcaaataaatttgcaaaaaccaaatactttttagtatatgtgctgccgaagcgagcacaaaaaccaaatactttttctgttatgttttaaaaatgatatctatctattaaatgcattcgatgtatttctatctcagcacactaccctttgcctttccaagtctttcaagacctctttccctttagggtcctcctttccacttgatgtgcacttgggtccaattaaagatgtccttatgtgtgtgtatatagcaatctatgtgagcatgggcagtcttccaagcaagttactacatctctgaagaaagccaacccaactcaccttcccctaataaccctcccctgggccctcatgtgcatctctctgctccgggctagactgtggactgacttgatgatgtgccggtctcagagctgtagatgtcatgaggtcctgagtgtaacacttgtggaagacgctgcttgacagcagtcctccccattccctagctcttacagtcagtgcaccacctcttcagtcatccttgagtgtgagagggaggcagtgtgacaaagatagcgactttggggcttgaaacttcaccatcatttattccctaaactttgactagctgtgggtctctatattaatgtctgtccactataaaaggaacttctctaatgaggagtgaaggctgcaccaagccaccaagaatctgtattgacatgagtttaatagaaaaagagaattaaagttaactttaattgtagcttaaccttttatgtataattttaatctgaaactgaacatccaatttaaagtggtccattggaaaagatagttgataaattatttcaataatatacattatttattaagcacgtgactatgaagttcacaaccatgctgctctgtgatagaagctaaagacaaaaaatatttttatgggagaaaatatcactgtttagtcttggctaacctggaattccctataggccaatctgtctttggactcatagagcctctgctgtccaagtgctgtaatcaagggcatgtgccatcatacctgggaaaaacagtcctgcatatagtcttgtatatacagtttgcattgaataaaaacagagtgggcaattaattgaaagcaccacctttactagctgcagcaacattttctgtcaaagaaaagcatctcaaccaaacccctgtgtctggagcttgttcggacaatatagggagagggaattggcttgtgttgggatttcttttgtactgctgtgttactttgcaattgtaaaaaaaaagaaatgacataaaggtggtgttttatatttgaatattcatataggtaaatggaatttggtttatcttttattttgttattttaagttgcctattagttttgtaaatagatcttattgttagaggtggtggtatttctcatgccatggcatgcacatggaagtcagaggacttgccaatactctcctcctgccaggatctccttctgtgtgattagagatggaattcatcttttcggtcttggtctgagcacctgaactcactaaggcatcacctaagcccagttatacttagtactttggcttagtttctctgatatactttgtttttcagcttctccatgggtggataatagtagaactccaaacaagattgatctatatgatgtacgcagaagaccatggtatgatgttttgactacctctaaagatgcgatcaataaatgggggttctatcatttgttaatacattgaggggaaacttccccttagtgggatgggtactctcttatatagaatgctgtgatactttcaaaatacaaatacaattaatgacaaatgaaaatgatgtgtttgagaaggaaatctctgtgaatactgataaaatgattttcagactcaattcaaatggattgctgtaagtcttctgtccagttttagctttcagtctcctttagctggcagggcttttcttcaaagagcaactactttgcttggggaaaagccctcacatagagatggcatggagtcacagacagcttgcctctggcagtgaactaatgggaactggccctaagctaacagtgctgcctagatacttccataaagtttaggttttattatttctccccaaagtaattttatttgtttgacactattaacgtaattcatcatcctaattcaaaacacaagaaaatatgaacaatatatgtttgaactctctgggagacaactatgaaacttcatgtatttttattattgattggattttgagtgtataaaagttatacttaaattgtaaatatactatttttaatgagttcacatatacaacatggtaccttatactctgttcttttcatattaatttttgagtatgtagtaatgaaaaggaatacttaaaatccctttaataaaggctatctgttcacttatttaagaaagtccttattgtgtttgtgcattgcttccaacttctggtattttcacacaaattctgtgcagcatagtggtggttgaaagaacatttagttatggctttcaagtgtcctgttgagtattcaataggtatttgcatgaacacaaccttaagtaatagttaccaatgtattagtgacttttaagtgtccttctgaatagccaatagcaatatacatgaacaaaaccttgaatactattaataatatttctctagtctattatttaatgaaagcaaatactatgttataacatagttgtacttaggaaacggtcattttatgggaactgtaggagcgaggtgtcactcatctgaagcagacactttgctaatcttttcatctgccactgagcagcacaccaggacaatgtgttaagatgctagacatgagctgggttgtcttgcatgggcaaacagtggagctttgcctattggtctgaatacagagcatggagtgggttgcaggcaaggcgctggagtgctcattctacaaataatcccaagtagtagcagctgtgctccgaaatgctgagaatgaattggcttgctttctgtctgttgctgggtcacttgagtcagatgcagcccttcccactgtggaaggcagatgcttaatcccttcaatttgttttgttcccctccagtctctggctaagcaatatgtgtcttctctttgttccttttctttctttttcttttcttttaaattactttgctgctatcaaatttagctctctacaagcacttaacttcctgcctaaatctcccgaagtgtgtatttcaaatacagagaatcagagatgtggttagagtttggaatttacatttaaactgtttctctggtgattcttggatgccataaatcttttagagtttctgcttcatatttctggtaatagtaatatccattacctggtttcagtttttaacttacctgttatatttccttttaatttgctttatcttaaggctttttgactacttatctctctgtgtttatttattttttgtatttaaaaatgtcataatttgttgttcactacaactgaaatgtcagcaatcagaatttaggggccgggcattgtggcatacgtctttaatcccagcacttgggaggcagaggcaggcgaatttctgagttcaaggccagcctggtctacagagtgagttccaggacagccagggttatacagagaaaccctgtctcgaaaaaacataaataaataaataaataaataatcggaatttaggattgttccttcttcagaagtaaataatcgtttggaaagtaattttagtcactgcttacattaaatttcatctttatttttttcataaatccctagaaactatgcattcaaaagccataatgtattaatatctctaatgggatcttaaatactcttttcaaaaaaacatttcagagtatgtttaaaagagcattgtttactatggtgtaatagaatgggactttgttttaatcatatcatatttcagaaatgtataaatatgaacacagcaaatgtcaatgttatacatagaggaaaaggcaatgttttgctggttctgtttcaggtacatccagggagctgcatcccccaaggacatgctcattctggtgaacgtgtgagtagtgcatgagacatttgtaggtaataataggttgttgggaattagaagtgtatgaaagttgggataacaaagaatcaatataatggttgtatatctagaaaaacaagccaaacttcgatattaacaattgattgttgacttggctcaatcagtaaagtgcttgttgcacaagaggaaggtctgagtgcatagctctggcttttacacgaaaacaattatataataagggagcagtggttgtgcctgtaatgccaattgtgaatagtaggagagaagaagtgccccagggctcactgattagacagtctaagggatcagtctgttctgggattactgtgagaccccagtattaaagaatatggtagagagccacagaagatacctggcattgacccctgacatccacacacatacactcacccacccattccacaccccacatttatgcaaatgggcacacgaagacaataacaaaaccagaaagatcattctattgaaaatgtaaataatgtatctgtatcaaactgggaaattatactattctaaaagctaatgagatcacttttgttctttatgatcgtgtttgataaatgacaggtgaatgacacacggacccatttgtctgtttgtaggagtggaagtgtgagcggattgactctgaaactcatccgaacatctgtctccgagatgttagaaaccctctctgatgatgatttcgtgaatgtagcttcagtgagtatggatggctgctccttctggaatcccgcctgcctgtctccctacatcattacatactgggttgtgttttccttcgatgggctgaggcattgtcagagtgctataagctgtcttactgatctagcctgtattgtcctttctgagatgcttagcattaccaccactgaagtaatgaatactgaatgaaaagcattccgtcccttccctcaaaggagcccacatgggtactgtgagcccttccacacacaggcacctttgggacatcttatgaagtagcatgattgaactgttaattgcttgtagctggttatactcatttgtgggctggacagctgaccttattgcactgagtttttcctaacagaagaaagtaaaggggaaagtgaataagctgggaattatcttagaccttttcctagctaggaagtactggccttgcggcaaatgggccaagatgtgccatgtctcatcttgatctatgtaatttagggacccaatttcatggccattttctagtgcaggaaatttcatatgtgaagtggttcatttatggagatgtggctgaaggagcagtagattttggtggtgttgatttttattgaacaaaggagaatagatctgcttctaaaaatcactgggtagggtgaagttaaagtctacaactgattgtctcactttcctggaattgggggaaacagaaatctccataatcctaattcttctaatgatgatggaatggtgatgctaagatggagaggatttatctctaaaatgaaatcactaagatagtcctgaatgctggtatggataaagagtatagaacccactatggataatgtaagacttcctggatagtgtgtcacgatgtataattctcaaggagaataattttaaaatattctatagttcaaataaattcagatggaaccttgtcaaactcttgttgagacattatggcaagatgaccacttcaattaagatataggaaagagcttcatttgtatataaatgtatcattttaaaaaaaaaactttggaatagcttaaaatgttatccttggcagaatgagagtggctcaaatatatttagttcagatttgtcatatataataaaaattatttaagacaagaaaataattaatactattaatgttttctttggcttctttcaaaaactgaacgtcagtctgagttgaagtagctcctttggaaatcacagggaatagaactggttcaacagtgagaggtgaagtgtgcaaagggagaatctaattggatattcttcatttaatttaaggaaaccaagtttgttgagaaattctagaaacctccaggaatagtcttttttttagagttgctaagctacaaaccctaaagacccagtgtgttaggtaccagcctcccttatgtgtagatgaacagaatatggccacgagcaggtattctgaactgggaaaattatggattctgaatgttgaaagctgatcagtgcataccctagtcaatgaatcagccataaaaattgacattatcacataataataataataataataataataataataataataataaacaataaacaactgtgatgatgatgatgatacacagaacattttataagagggtggtgagcttaaagttagtgtctgaggggaatccatgcagctgttctatagacaagtaagtggcacaaagaggtaataatgaaaactggtacagactgacttcaacctggatc

SEQ ID NO: 6 - DNA, 72 bp, Mus musculus
gcttctccatgggtggataatagtagaactccaaacaagattgatctatatgatgtacgcagaagaccatgg

SEQ ID NO: 7 - DNA, 46 bp, Artificial
  Synthetic oligonucleotide
caaaacatcataccatggtgctctgcgtacatcatatagatcaatc

SEQ ID NO: 8 - DNA, 221 bp, Artificial
  Synthetic oligonucleotide
ggtggtgttttatatttgaatattcatataggtaaatggaatttggtttatcttttattttgttattttaagttgcctattagttttgtaaatagatcttattgttagaggtggtggtatttctcatgccatggcatgcacatggaagtcagaggacttgccaatactctcctcctgccaggatctccttctgtgtgattagagatggaattcatcttttc

SEQ ID NO: 9 - DNA, 180 bp, Artificial
  Synthetic oligonucleotide
ggtcttggtctgagcacctgaactcactaaggcatcacctaagcccagttatacttagtactttggcttagtttctctgatatactttgtttttcagcttctccatgggtggataatagtagaactccaaacaagattgatctatatgatgtacgcagagcaccatggtatgatgttttg

SEQ ID NO: 10 - DNA, 2101 bp, Saccharomyces cerevisiae
ggccatagcggccggatcctcgaggcgcgccatttaaatcgttggcagaacatatccatcgcgtccgccatctccagcagccgcacgcggcgcatccccccccccctttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattactcgggcgtaatgatactaaatcggaaccctaaagggagcccccgatttagagcttgacggggaaagccgggctgcagtgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttatttaaatggcgcgccggccagcgaggcc

SEQ ID NO: 11 - DNA, 20 bp, Artificial
  Synthetic oligonucleotide
gcagcgcatcgccttctatc

SEQ ID NO: 12 - DNA, 22 bp, Artificial
  Synthetic oligonucleotide
ctctttgaagaaaagccctgcc

SEQ ID NO: 13 - DNA, 24 bp, Artificial
  Synthetic oligonucleotide
ctaatgccaggtggtttttaatcc

SEQ ID NO: 14 - DNA, 21 bp, Artificial
  Synthetic oligonucleotide
ctgatctgcattattggtggg

SEQ ID NO: 15 - DNA, 22 bp, Artificial
  Synthetic oligonucleotide
cattttgtaggtgtgttgactc

SEQ ID NO: 16 - DNA, 25 bp, Artificial
  Synthetic oligonucleotide
ctcaacaatgactcataattgattg

SEQ ID NO: 17 - protein, 24 aa, Mus musculus
ASPWVDNSRTPNKIDLYDVRRAPW

SEQ ID NO: 18 - protein, 41 aa, Mus musculus
ASPWVDNSRTPNKIDLYDVRRAPWYIQGAASPKDMLILVNV

SEQ ID NO: 19 - protein, 74 aa, Mus musculus
ASPWVDNSRTPNKIDLYDVRRAPWYIQGAASPKDMLILVNVSGSVSGLTLKLIRTSVSEMLETLSDDDFVNVAS

SEQ ID NO: 20 - DNA, 72 bp, Mus musculus
gcttctccatgggtggataatagtagaactccaaacaagattgatctatatgatgtacgcagagcaccatgg

SEQ ID NO: 21 - DNA, 2492 bp, Mus musculus
gcttctccatgggtggataatagtagaactccaaacaagattgatctatatgatgtacgcagagcaccatggtatgatgttttgactacctctaaagatgcgatcaataaatgggggttctatcatttgttaatacattgaggggaaacttccccttagtgggatgggtactctcttatatagaatgctgtgatactttcaaaatacaaatacaattaatgacaaatgaaaatgatgtgtttgagaaggaaatctctgtgaatactgataaaatgattttcagactcaattcaaatggattgctgtaagtcttctgtccagttttagctttcagtctcctttagctggcagggcttttcttcaaagagcaactactttgcttggggaaaagccctcacatagagatggcatggagtcacagacagcttgcctctggcagtgaactaatgggaactggccctaagctaacagtgctgcctagatacttccataaagtttaggttttattatttctccccaaagtaattttatttgtttgacactattaacgtaattcatcatcctaattcaaaacacaagaaaatatgaacaatatatgtttgaactctctgggagacaactatgaaacttcatgtatttttattattgattggattttgagtgtataaaagttatacttaaattgtaaatatactatttttaatgagttcacatatacaacatggtaccttatactctgttcttttcatattaatttttgagtatgtagtaatgaaaaggaatacttaaaatccctttaataaaggctatctgttcacttatttaagaaagtccttattgtgtttgtgcattgcttccaacttctggtattttcacacaaattctgtgcagcatagtggtggttgaaagaacatttagttatggctttcaagtgtcctgttgagtattcaataggtatttgcatgaacacaaccttaagtaatagttaccaatgtattagtgacttttaagtgtccttctgaatagccaatagcaatatacatgaacaaaaccttgaatactattaataatatttctctagtctattatttaatgaaagcaaatactatgttataacatagttgtacttaggaaacggtcattttatgggaactgtaggagcgaggtgtcactcatctgaagcagacactttgctaatcttttcatctgccactgagcagcacaccaggacaatgtgttaagatgctagacatgagctgggttgtcttgcatgggcaaacagtggagctttgcctattggtctgaatacagagcatggagtgggttgcaggcaaggcgctggagtgctcattctacaaataatcccaagtagtagcagctgtgctccgaaatgctgagaatgaattggcttgctttctgtctgttgctgggtcacttgagtcagatgcagcccttcccactgtggaaggcagatgcttaatcccttcaatttgttttgttcccctccagtctctggctaagcaatatgtgtcttctctttgttccttttctttctttttcttttcttttaaattactttgctgctatcaaatttagctctctacaagcacttaacttcctgcctaaatctcccgaagtgtgtatttcaaatacagagaatcagagatgtggttagagtttggaatttacatttaaactgtttctctggtgattcttggatgccataaatcttttagagtttctgcttcatatttctggtaatagtaatatccattacctggtttcagtttttaacttacctgttatatttccttttaatttgctttatcttaaggctttttgactacttatctctctgtgtttatttattttttgtatttaaaaatgtcataatttgttgttcactacaactgaaatgtcagcaatcagaatttaggggccgggcattgtggcatacgtctttaatcccagcacttgggaggcagaggcaggcgaatttctgagttcaaggccagcctggtctacagagtgagttccaggacagccagggttatacagagaaaccctgtctcgaaaaaacataaataaataaataaataaataatcggaatttaggattgttccttcttcagaagtaaataatcgtttggaaagtaattttagtcactgcttacattaaatttcatctttatttttttcataaatccctagaaactatgcattcaaaagccataatgtattaatatctctaatgggatcttaaatactcttttcaaaaaaacatttcagagtatgtttaaaagagcattgtttactatggtgtaatagaatgggactttgttttaatcatatcatatttcagaaatgtataaatatgaacacagcaaatgtcaatgttatacatagaggaaaaggcaatgttttgctggttctgtttcaggtacatccagggagctgcatcccccaaggacatgctcattctggtgaacgtg

SEQ ID NO: 22 - DNA, 123 bp, Mus musculus
gcttctccatgggtggataatagtagaactccaaacaagattgatctatatgatgtacgcagagcaccatggtacatccagggagctgcatcccccaaggacatgctcattctggtgaacgtg

SEQ ID NO: 23 - DNA, 3529 bp, Mus musculus
gcttctccatgggtggataatagtagaactccaaacaagattgatctatatgatgtacgcagagcaccatggtatgatgttttgactacctctaaagatgcgatcaataaatgggggttctatcatttgttaatacattgaggggaaacttccccttagtgggatgggtactctcttatatagaatgctgtgatactttcaaaatacaaatacaattaatgacaaatgaaaatgatgtgtttgagaaggaaatctctgtgaatactgataaaatgattttcagactcaattcaaatggattgctgtaagtcttctgtccagttttagctttcagtctcctttagctggcagggcttttcttcaaagagcaactactttgcttggggaaaagccctcacatagagatggcatggagtcacagacagcttgcctctggcagtgaactaatgggaactggccctaagctaacagtgctgcctagatacttccataaagtttaggttttattatttctccccaaagtaattttatttgtttgacactattaacgtaattcatcatcctaattcaaaacacaagaaaatatgaacaatatatgtttgaactctctgggagacaactatgaaacttcatgtatttttattattgattggattttgagtgtataaaagttatacttaaattgtaaatatactatttttaatgagttcacatatacaacatggtaccttatactctgttcttttcatattaatttttgagtatgtagtaatgaaaaggaatacttaaaatccctttaataaaggctatctgttcacttatttaagaaagtccttattgtgtttgtgcattgcttccaacttctggtattttcacacaaattctgtgcagcatagtggtggttgaaagaacatttagttatggctttcaagtgtcctgttgagtattcaataggtatttgcatgaacacaaccttaagtaatagttaccaatgtattagtgacttttaagtgtccttctgaatagccaatagcaatatacatgaacaaaaccttgaatactattaataatatttctctagtctattatttaatgaaagcaaatactatgttataacatagttgtacttaggaaacggtcattttatgggaactgtaggagcgaggtgtcactcatctgaagcagacactttgctaatcttttcatctgccactgagcagcacaccaggacaatgtgttaagatgctagacatgagctgggttgtcttgcatgggcaaacagtggagctttgcctattggtctgaatacagagcatggagtgggttgcaggcaaggcgctggagtgctcattctacaaataatcccaagtagtagcagctgtgctccgaaatgctgagaatgaattggcttgctttctgtctgttgctgggtcacttgagtcagatgcagcccttcccactgtggaaggcagatgcttaatcccttcaatttgttttgttcccctccagtctctggctaagcaatatgtgtcttctctttgttccttttctttctttttcttttcttttaaattactttgctgctatcaaatttagctctctacaagcacttaacttcctgcctaaatctcccgaagtgtgtatttcaaatacagagaatcagagatgtggttagagtttggaatttacatttaaactgtttctctggtgattcttggatgccataaatcttttagagtttctgcttcatatttctggtaatagtaatatccattacctggtttcagtttttaacttacctgttatatttccttttaatttgctttatcttaaggctttttgactacttatctctctgtgtttatttattttttgtatttaaaaatgtcataatttgttgttcactacaactgaaatgtcagcaatcagaatttaggggccgggcattgtggcatacgtctttaatcccagcacttgggaggcagaggcaggcgaatttctgagttcaaggccagcctggtctacagagtgagttccaggacagccagggttatacagagaaaccctgtctcgaaaaaacataaataaataaataaataaataatcggaatttaggattgttccttcttcagaagtaaataatcgtttggaaagtaattttagtcactgcttacattaaatttcatctttatttttttcataaatccctagaaactatgcattcaaaagccataatgtattaatatctctaatgggatcttaaatactcttttcaaaaaaacatttcagagtatgtttaaaagagcattgtttactatggtgtaatagaatgggactttgttttaatcatatcatatttcagaaatgtataaatatgaacacagcaaatgtcaatgttatacatagaggaaaaggcaatgttttgctggttctgtttcaggtacatccagggagctgcatcccccaaggacatgctcattctggtgaacgtgtgagtagtgcatgagacatttgtaggtaataataggttgttgggaattagaagtgtatgaaagttgggataacaaagaatcaatataatggttaggttgttgggaattagaagtgtatgaaagttgggataacaaagaatcaatataatggttgtatatctagaaaaacaagccaaacttcgatattaacaattgattgttgacttggctcatgtatatctagaaaaacaagccaaacttcgatattaacaattgattgttgacttggctcaatcagtaaagtgcttgttgcacaagaggaaggtctgagtgcatagctctggcttttacacatcagtaaagtgcttgttgcacaagaggaaggtctgagtgcatagctctggcttttacacgaaaacaattatataataagggagcagtggttgtgcctgtaatgccaattgtgaatagtagaaaacaattatataataagggagcagtggttgtgcctgtaatgccaattgtgaatagtaggagagaagaagtgccccagggctcactgattagacagtctaagggatcagtctgttctgggagagaagaagtgccccagggctcactgattagacagtctaagggatcagtctgttctgggattactgtgagaccccagtattaaagaatatggtagagagccacagaagatacctggcattgacccctgacatccacacacatacactcacccacccattccacaccccacatttatgcaaatgggcacacgaagacaataacaaaaccagaaagatcattctattgaaaatgtaaataatgtatctgtatcaaactgggaaattatactattctaaaagctaatgagatcacttttgttctttatgatcgtgtttgataaatgacaggtgaatgacacacggacccatttgtctgtttgtaggagtggaagtgtgagcggattgactctgaaactcatccgaacatctgtctccgagatgttagaaaccctctctgatgatgatttcgtgaatgtagcttca

SEQ ID NO: 24 - DNA, 222 bp, Mus musculus
gcttctccatgggtggataatagtagaactccaaacaagattgatctatatgatgtacgcagagcaccatggtacatccagggagctgcatcccccaaggacatgctcattctggtgaacgtgagtggaagtgtgagcggattgactctgaaactcatccgaacatctgtctccgagatgttagaaaccctctctgatgatgatttcgtgaatgtagcttca

SEQ ID NO: 25 - protein, 1103 aa, Mus musculus
MAAGCLLALTLTLFQSGLIGPSSEEPFPSPVTIKSWVDKMQEDLVTLAKTASGVTQLADIYEKYQDLYTVEPNNARQLVEIAARDIEKLLSNRSKALVRLAMEAEKVQAAHQWREDFASNEVVYYNAKDDLDPERNESEPGSQRIKPVFIEDANFGRQISYQHAAVHIPTDIYEGSTIVLNELNWTSALDEVFKRNRDEDPTLLWQVFGSATGLARYYPASPWVDNSRTPNKIDLYDVRRRPWYIQGAASPKDMLILVDVSGSVSGLTLKLIRTSVSEMLETLSDDDFVNVASFNSNAQDVSCFQHLVQANVRNKKVLKDAVNNITAKGITDYKKGFSFAFEQLLNYNVSRANCNKIIMLFTDGGEERAQEIFAKYNKDKKVRVFTFSVGQHNYDRGPIQWMACENKGYYYEIPSIGAIRINTQEYLDVLGRPMVLAGDKAKQVQWTNVYLDALELGLVITGTLPVFNVTGQSENKTNLKNQLILGVMGVDVSLEDIKRLTPRFTLCPNGYYFAIDPNGYVLLHPNLQPKPIGVGIPTINLRKRRPNVQNPKSQEPVTLDFLDAELENEIKVEIRNKMIDGESGEKTFRTLVKSQDERYIDKGNRTYTWTPVNGTDYSLALVLPTYSFYYIKAKLEETITQARYSETLKPDNFEESGYTFIAPREYCNDLKPSDNNTEFLLNFNEFIDRKTPNNPSCNTDLINRILLDAGFTNELVQNYWSKQKNIKGVKARFVVTDGGITRVYPKEAGENWQENPETYEDSFYKRSLDNDNYVFTAPYFNKSGPGAYESGIMVSKAVELYIQGKLLKPAVVGIKIDVNSWIENFTKTSIRDPCAGPVCDCKRNSDVMDCVILDDGGFLLMANHDDYTNQIGRFFGEIDPSMMRHLVNISLYAFNKSYDYQSVCDPGAAPKQGAGHRSAYVPSIADILQIGWWATAAAWSILQQLLLSLTFPRLLEAVEMEEDDFTASLSKQSCITEQTQYFFKNDTKSFSGLLDCGNCSRIFHVEKLMNTNLVFIMVESKGTCPCDTRLLMQAEQTSDGPDPCDMVKQPRYRKGPDVCFDNNVLEDYTDCGGVSGLNPSLWSIFGLQFILLWLVSGSRHYLL

SEQ ID NO: 26 - protein, 1091 aa, Mus musculus
MAAGCLLALTLTLFQSGLIGPSSEEPFPSPVTIKSWVDKMQEDLVTLAKTASGVTQLADIYEKYQDLYTVEPNNARQLVEIAARDIEKLLSNRSKALVRLAMEAEKVQAAHQWREDFASNEVVYYNAKDDLDPERNESEPGSQRIKPVFIEDANFGRQISYQHAAVHIPTDIYEGSTIVLNELNWTSALDEVFKRNRDEDPTLLWQVFGSATGLARYYPASPWVDNSRTPNKIDLYDVRRRPWYIQGAASPKDMLILVDVSGSVSGLTLKLIRTSVSEMLETLSDDDFVNVASFNSNAQDVSCFQHLVQANVRNKKVLKDAVNNITAKGITDYKKGFSFAFEQLLNYNVSRANCNKIIMLFTDGGEERAQEIFAKYNKDKKVRVFTFSVGQHNYDRGPIQWMACENKGYYYEIPSIGAIRINTQEYLDVLGRPMVLAGDKAKQVQWTNVYLDALELGLVITGTLPVFNVTGQSENKTNLKNQLILGVMGVDVSLEDIKRLTPRFTLCPNGYYFAIDPNGYVLLHPNLQPKNPKSQEPVTLDFLDAELENEIKVEIRNKMIDGESGEKTFRTLVKSQDERYIDKGNRTYTWTPVNGTDYSLALVLPTYSFYYIKAKLEETITQARSKKGKMKDSETLKPDNFEESGYTFIAPREYCNDLKPSDNNTEFLLNFNEFIDRKTPNNPSCNTDLINRILLDAGFTNELVQNYWSKQKNIKGVKARFVVTDGGITRVYPKEAGENWQENPETYEDSFYKRSLDNDNYVFTAPYFNKSGPGAYESGIMVSKAVELYIQGKLLKPAVVGIKIDVNSWIENFTKTSIRDPCAGPVCDCKRNSDVMDCVILDDGGFLLMANHDDYTNQIGRFFGEIDPSMMRHLVNISLYAFNKSYDYQSVCDPGAAPKQGAGHRSAYVPSIADILQIGWWATAAAWSILQQLLLSLTFPRLLEAVEMEEDDFTASLSKQSCITEQTQYFFKNDTKSFSGLLDCGNCSRIFHVEKLMNTNLVFIMVESKGTCPCDTRLLMQAEQTSDGPDPCDMVKQPRYRKGPDVCFDNNVLEDYTDCGGVSGLNPSLWSIFGLQFILLWLVSGSRHYLL

SEQ ID NO: 27 - protein, 1086 aa, Mus musculus
MAAGCLLALTLTLFQSGLIGPSSEEPFPSPVTIKSWVDKMQEDLVTLAKTASGVTQLADIYEKYQDLYTVEPNNARQLVEIAARDIEKLLSNRSKALVRLAMEAEKVQAAHQWREDFASNEVVYYNAKDDLDPERNESEPGSQRIKPVFIEDANFGRQISYQHAAVHIPTDIYEGSTIVLNELNWTSALDEVFKRNRDEDPTLLWQVFGSATGLARYYPASPWVDNSRTPNKIDLYDVRRRPWYIQGAASPKDMLILVDVSGSVSGLTLKLIRTSVSEMLETLSDDDFVNVASFNSNAQDVSCFQHLVQANVRNKKVLKDAVNNITAKGITDYKKGFSFAFEQLLNYNVSRANCNKIIMLFTDGGEERAQEIFAKYNKDKKVRVFTFSVGQHNYDRGPIQWMACENKGYYYEIPSIGAIRINTQEYLDVLGRPMVLAGDKAKQVQWTNVYLDALELGLVITGTLPVFNVTGQSENKTNLKNQLILGVMGVDVSLEDIKRLTPRFTLCPNGYYFAIDPNGYVLLHPNLQPKEPVTLDFLDAELENEIKVEIRNKMIDGESGEKTFRTLVKSQDERYIDKGNRTYTWTPVNGTDYSLALVLPTYSFYYIKAKLEETITQARSKKGKMKDSETLKPDNFEESGYTFIAPREYCNDLKPSDNNTEFLLNFNEFIDRKTPNNPSCNTDLINRILLDAGFTNELVQNYWSKQKNIKGVKARFVVTDGGITRVYPKEAGENWQENPETYEDSFYKRSLDNDNYVFTAPYFNKSGPGAYESGIMVSKAVELYIQGKLLKPAVVGIKIDVNSWIENFTKTSIRDPCAGPVCDCKRNSDVMDCVILDDGGFLLMANHDDYTNQIGRFFGEIDPSMMRHLVNISLYAFNKSYDYQSVCDPGAAPKQGAGHRSAYVPSIADILQIGWWATAAAWSILQQLLLSLTFPRLLEAVEMEEDDFTASLSKQSCITEQTQYFFKNDTKSFSGLLDCGNCSRIFHVEKLMNTNLVFIMVESKGTCPCDTRLLMQAEQTSDGPDPCDMVKQPRYRKGPDVCFDNNVLEDYTDCGGVSGLNPSLWSIFGLQFILLWLVSGSRHYLL

SEQ ID NO: 28 - protein, 1079 aa, Mus musculus
MAAGCLLALTLTLFQSGLIGPSSEEPFPSPVTIKSWVDKMQEDLVTLAKTASGVTQLADIYEKYQDLYTVEPNNARQLVEIAARDIEKLLSNRSKALVRLAMEAEKVQAAHQWREDFASNEVVYYNAKDDLDPERNESEPGSQRIKPVFIEDANFGRQISYQHAAVHIPTDIYEGSTIVLNELNWTSALDEVFKRNRDEDPTLLWQVFGSATGLARYYPASPWVDNSRTPNKIDLYDVRRRPWYIQGAASPKDMLILVDVSGSVSGLTLKLIRTSVSEMLETLSDDDFVNVASFNSNAQDVSCFQHLVQANVRNKKVLKDAVNNITAKGITDYKKGFSFAFEQLLNYNVSRANCNKIIMLFTDGGEERAQEIFAKYNKDKKVRVFTFSVGQHNYDRGPIQWMACENKGYYYEIPSIGAIRINTQEYLDVLGRPMVLAGDKAKQVQWTNVYLDALELGLVITGTLPVFNVTGQSENKTNLKNQLILGVMGVDVSLEDIKRLTPRFTLCPNGYYFAIDPNGYVLLHPNLQPKEPVTLDFLDAELENEIKVEIRNKMIDGESGEKTFRTLVKSQDERYIDKGNRTYTWTPVNGTDYSLALVLPTYSFYYIKAKLEETITQARYSETLKPDNFEESGYTFIAPREYCNDLKPSDNNTEFLLNFNEFIDRKTPNNPSCNTDLINRILLDAGFTNELVQNYWSKQKNIKGVKARFVVTDGGITRVYPKEAGENWQENPETYEDSFYKRSLDNDNYVFTAPYFNKSGPGAYESGIMVSKAVELYIQGKLLKPAVVGIKIDVNSWIENFTKTSIRDPCAGPVCDCKRNSDVMDCVILDDGGFLLMANHDDYTNQIGRFFGEIDPSMMRHLVNISLYAFNKSYDYQSVCDPGAAPKQGAGHRSAYVPSIADILQIGWWATAAAWSILQQLLLSLTFPRLLEAVEMEEDDFTASLSKQSCITEQTQYFFKNDTKSFSGLLDCGNCSRIFHVEKLMNTNLVFIMVESKGTCPCDTRLLMQAEQTSDGPDPCDMVKQPRYRKGPDVCFDNNVLEDYTDCGGVSGLNPSLWSIFGLQFILLWLVSGSRHYLL

SEQ ID NO: 29 - protein, 1084 aa, Mus musculus
MAAGCLLALTLTLFQSGLIGPSSEEPFPSPVTIKSWVDKMQEDLVTLAKTASGVTQLADIYEKYQDLYTVEPNNARQLVEIAARDIEKLLSNRSKALVRLAMEAEKVQAAHQWREDFASNEVVYYNAKDDLDPERNESEPGSQRIKPVFIEDANFGRQISYQHAAVHIPTDIYEGSTIVLNELNWTSALDEVFKRNRDEDPTLLWQVFGSATGLARYYPASPWVDNSRTPNKIDLYDVRRRPWYIQGAASPKDMLILVDVSGSVSGLTLKLIRTSVSEMLETLSDDDFVNVASFNSNAQDVSCFQHLVQANVRNKKVLKDAVNNITAKGITDYKKGFSFAFEQLLNYNVSRANCNKIIMLFTDGGEERAQEIFAKYNKDKKVRVFTFSVGQHNYDRGPIQWMACENKGYYYEIPSIGAIRINTQEYLDVLGRPMVLAGDKAKQVQWTNVYLDALELGLVITGTLPVFNVTGQSENKTNLKNQLILGVMGVDVSLEDIKRLTPRFTLCPNGYYFAIDPNGYVLLHPNLQPKNPKSQEPVTLDFLDAELENEIKVEIRNKMIDGESGEKTFRTLVKSQDERYIDKGNRTYTWTPVNGTDYSLALVLPTYSFYYIKAKLEETITQARYSETLKPDNFEESGYTFIAPREYCNDLKPSDNNTEFLLNFNEFIDRKTPNNPSCNTDLINRILLDAGFTNELVQNYWSKQKNIKGVKARFVVTDGGITRVYPKEAGENWQENPETYEDSFYKRSLDNDNYVFTAPYFNKSGPGAYESGIMVSKAVELYIQGKLLKPAVVGIKIDVNSWIENFTKTSIRDPCAGPVCDCKRNSDVMDCVILDDGGFLLMANHDDYTNQIGRFFGEIDPSMMRHLVNISLYAFNKSYDYQSVCDPGAAPKQGAGHRSAYVPSIADILQIGWWATAAAWSILQQLLLSLTFPRLLEAVEMEEDDFTASLSKQSCITEQTQYFFKNDTKSFSGLLDCGNCSRIFHVEKLMNTNLVFIMVESKGTCPCDTRLLMQAEQTSDGPDPCDMVKQPRYRKGPDVCFDNNVLEDYTDCGGVSGLNPSLWSIFGLQFILLWLVSGSRHYLL

SEQ ID NO: 30 - protein, 1091 aa, Rattus norvegicus
MAAGCLLALTLTLFQSWLIGPSSEEPFPSPVTIKSWVDKMQEDLVTLAKTASGVTQLADIYEKYQDLYTVEPNNARQLVEIAARDIEKLLSNRSKALVRLAMEAEKVQAAHQWREDFASNEVVYYNAKDDLDPERNESESGSQRIKPVFIEDANFGRQISYQHAAVHIPTDIYEGSTIVLNELNWTSALDEVFKRNRDEDPTLLWQVFAADRLARYYPASPWVDNSRTPNKIDLYDVRRRPWYIQGAASPKDMLILVDVSGSVSGLTLKLIRTSVSEMLETLSDDDFVNVASFNSNAQDVSCFQHLVQANVRNKKVLKDAVNNITAKGITDYKKGFTFAFEQLLNYNVSRANCNKIIMLFTDGGEERAQEIFAKYNKDKKVRVFTFSVGQHNYDRGPIQWMACENKGYYYEIPSIGAIRINTQEYLDVLGRPMVLAGDKAKQVQWTNVYLDALELGLVITGTLPVFNVTGQSENKTNLKNQLILGVMGVDVSLEDIKRLTPRFTLCPNGYYFAIDPNGYVLLHPNLQPKNPKSQEPVTLDFLDAELENDIKVEIRNKMIDGESGEKTFRTLVKSQDERYIDKGNRTYTWTPVNGTDYRYLALVLPTYSFYYIKAKIEETITQARSKKGKMKDSETLKPDNFEESGYTFIAPREYCNDLKPSDNNTEFLLNFNEFIDRKTPNNPSCNTDLINRILLDAGFTNELVQNYWSKQKNIKGVKARFVVTDGGITRVYPKEAGENWQENPETYEDSFYKRSLDNDNYVFTAPYFNKSGPGAYESGIMVSKAVELYIQGKLLKPAVVGIKIDVNSWIENFTKTSIRDPCAGPVCDCKRNSDVMDCVILDDGGFLLMANHDDYTNQIGRFFGEIDPRMMRHLVNISLYAFNKSYDYQSVCDPGAAPKQGAGHRSAYVPSITDILQIGWWATAAAWSILQQLLLSLTFPRLLEAVEMEEDDFTASLSKQSCITEQTQYFFKNDTKSFSGLLDCGNCSRIFHVEKLMNTNLVFIMVESKGTCPCDTRLLMQAEQTSDGPDPCDMVKQPRYRKGPDVCFDNNVLEDYTDCGGVSGLNPSLWSIFGLQFILLWLVSGSRHYLW

SEQ ID NO: 31 - protein, 1091 aa, Sus scrofa
MAAGCLLALTLTLFQSLLIGPSSQEPFPSAVTIKSWVDKMQEDLVTLAKTASGVNQLVDIYEKYQDLYTVEPNNARQLVEIAARDIEKLLSNRSKALVRLALEAEKVQAAHQWREDFASNEVVYYNAKDDLDPEKNDSEPGSQRIKPVFIDDANFGRQISYQHAAVHIPTDIYEGSTIVLNELNWTSALDEVFKKNREEDPSLLWQVFGSATGLARYYPASPWVDNSRTPNKIDLYDVRRRPWYIQGAASPKDMLILVDVSGSVSGLTLKLIRTSVSEMLETLSDDDFVNVASFNSNAQDVSCFQHLVQANVRNKKVLKDAVNNITAKGITDYKKGFSFAFEQLLNYNVSRANCNKIIMLFTDGGEERAQEIFAKYNKDKKVRVFTFSVGQHNYDRGPIQWMACENKGYYYEIPSIGAIRINTQEYLDVLGRPMVLAGDKAKQVQWTNVYLDALELGLVITGTLPVFNITGQNENKTNLKNQLILGVMGVDVSLEDIKRLTPRFTLCPNGYYFAIDPNGYVLLHPNLQPKNPKSQEPVTLDFLDAELENDIKVEIRNKMIDGESGEKTFRTLVKSQDERYIDKGNRTYTWTPVNGTDYSLALVLPTYSFYYIKAKIEETITQARSKKGKMKDSETLKPDNFEESGYTFIAPRDYCNDLKISDNNTEFLLNFNEFIDRKTPNNPSCNTDLINRVLLDAGFTNELVQNYWSKQKNIKGVKARFVVTDGGITRVYPKEAGENWQENPETYEDSFYKRSLDNDNYVFTAPYFNKSGPGAYESGIMVSKAVEIYIQGKLLKPAVVGIKIDVNSWIENFTKTSIRDPCAGPVCDCKRNSDVMDCVILDDGGFLLMANHDDYTNQIGRFFGEIDPSLMRHLVNISVYAFNKSYDYQSVCEPGAAPKQGAGHRSAYVPSIADILHIGWWATAAAWSILQQFLLSLTFPRLLEAVEMEDDDFTASLSKQSCITEQTQYFFDNDSKSFSGVLDCGNCSRIFHVEKLMNTNLIFIMVESKGTCPCDTRLLIQAEQTSDGPDPCDMVKQPRYRKGPDVCFDNNALEDYTDCGGVSGLNPSLWSIFGIQCVLLWLLSGSRHYQL

(19) United States
(12) Patent Application Publication
(10) Pub. No.: US 2005/0044581 A1
(43) Pub. Date: Feb. 24, 2005

(54) Animals and cells containing a mutated alpha2/omega1 gene

(76) Inventors: Scott Phillip Baron, Ann Arbor, MI (US); Debra Lynn Hidayetoglu, West Bloomfield, MI (US); Margaret Ann Johns, Atlanta, GA (US); James David Offord, Dexter, MI (US); Ti-Zhi Su, Ann Arbor, MI (US)

Correspondence Address: WARNER-LAMBERT COMPANY, 2800 PLYMOUTH RD, ANN ARBOR, MI, 48105, US

(21) Appl. No.: 10/823,447

(22) Filed: Apr. 13, 2004

Related U.S. Application Data
(60) Provisional application No. 60/462,634, filed on Apr. 14, 2003.
(60) Provisional application No. 60/518,110, filed on Nov. 7, 2003.

(51) Int. Cl. A01K 67/027

(52) U.S. Cl. 800/14; 800/18

(57) ABSTRACT

The invention features non-human mammals and animal cells that contain a targeted disruption of a α2/δ1 gene.

### FIELD OF THE INVENTION

The present invention features genetically-modified non-human mammals and animal cells containing a mutated α2/δ1-subunit gene.

### BACKGROUND OF THE INVENTION

α2/δ1 is an auxiliary subunit of voltage sensitive calcium channels. This subunit is required for the proper functioning of these channels. α2/δ1 is known to have a role in epilepsy, anxiety, pain, analgesia, tinnitus, migraine prophylaxis, and hot flashes. See McLean M J et al. (1993) Neurol. 43:2292-2298; Field et al.(2001), Br. J. Pharmacol. 132(January):1-4; Mauri M C et al. (2001) Clin Drug Invest, 21(3): 169-74; Takasaki I et al. (2001) Pharmacol Exp Ther, 296(2)(February): 270-5; Bauer C A and Brozoski T J (2001) JARO 2(1)(March):54-64; Mathew N T et al. (2001) Headache 41(February): 119-28; and Loprinzi et al. (2002) Mayo Clin Proc, 77(11) (November): 1159-63. However, the pathophysiological role of the protein in these diseases remains unclear.

This protein is a member of a family which currently includes the four members α2/δ1 to α2/δ4. For example, see WO 00/20450, which refers to α2/δ1 to α2/δ4 as α2δ-A, α2δ-B, α2δ-C, and α2δ-D respectively. The Genbank accession numbers for the α2/δ1 subunit include: M76559 (human); U73483, U73484, U73485, U73486, and U73487 (mouse); M86621 (rat); and AF077665 (pig). See also Brown and N. Gee. (1998) J. Biol. Chem. 273:25458-25465.

The α2/δ1 and α2/δ2 proteins have been shown to bind the anticonvulsant 1-(aminomethyl)cyclohexaneacetic acid (gabapentin) with high affinity. In vitro functional assays have shown conflicting results with regard to the ability of gabapentin and other α2/δ1 ligands to have any functional effect on the channel activity. See Stefani A et al. (2001) Epilepsy Res; 43(3):239-48; and Ng G Y et al. (2001) Mol Pharmacol Jan; 59(1): 144-52. Binding of gabapentin to the porcine α2/δ1 subunit can be greatly reduced by the R217A mutation; that is by mutating a single amino acid at position 217 (position 241 in the polypeptide having its leader sequence), from an arginine to an alanine (Wang, M et al. (1999), Biochem. J. 342: 313-320).

Research tools, such as genetically-modified, non-human mammals including α2/δ1 mutant mice comprising R217A and R217A-like mutations, are useful for further defining the physiological role of α2/δ1 action; for further defining the therapeutic implications associated with the binding of α2/δ1 ligands to their binding site; for further testing the mechanism of action of α2/δ ligands in vivo by treating these mutant animals with the ligands and determining if the compounds are still effective, and/or retain the same characteristics; for determining the relative contribution of α2/δ1 polypeptide in mediating the effects of α2/δ ligands; for profiling compounds to determine α2/δ subtype selectivity of compounds; for identifying signal transduction pathways in which α2/δ ligands are involved; for testing the role of α2/δ in disease processes including anxiety, depression, schizophrenia, bipolar disease, and other disorders; testing the role of α2/δ in cardiovascular disease processes; and for using these mutant transgenic animals as breeding intermediates by breeding the animals with other animals, preferably with other α2/δ mutant animals; and for determining the role of leucine in affecting α2/δ activity (Brown J P, Dissanayake V U, Briggs A R, Milic M R, Gee NS (1998) Anal Biochem. Jan 15; 255(2):236-43).

### SUMMARY OF THE INVENTION

As stated above, α2/δ1 is known to have a role in disorders such as epilepsy, anxiety, pain and analgesia, tinnitus, migraine prophylaxis, and hot flashes. However, the pathophysiological role of the protein in these disorders remains unclear.

It is also known that knockout animals are valuable research tools for studying physiological and pathological roles of particular proteins. For example, see Steele, P M, J F Medina, W L Nores, and M D Mauk (1998) Cell 95 (7):879-882.

However, in the case of oα2/δ1, attempts to produce a complete α2/δ1 knockout animal have failed because such complete abolishment of α2/δ1 polypeptide/function is lethal. In this regard, deletion of exon 8 (eg. FIG. 1) generated an α2/δ1 protein that is non-functional with respect to calcium channel function. However, none of the animals born from heterozygous X heterozygous matings, in 4 generations, have shown the null (homozygous for the exon 8 deletion) genotype.

This invention encompasses a viable, genetically-modified, non-human mammal, comprising a mutated α2/δ1 gene. Those skilled in the art will fully understand the terms used herein in the description and the appendant claims to describe the present invention. Nonetheless, unless otherwise provided herein, the following terms are as described immediately below.

In one embodiment, the invention encompasses a viable, genetically-modified, non-human mammal, comprising an R217A-like mutation. In one embodiment, the mammal is a rodent; in another, a mouse; and in another, the mammal is homozygous for the R217A-like mutation. By “R217A-like mutation” is intended to mean a substitution or deletion of at least one nucleotide in a mammalian gene encoding an α2/δ1 polypeptide, wherein a genetically modified animal or animal cell comprising said gene having said substitution or deletion and expressing said polypeptide is viable and exhibits a phenotype in which the binding of gabapentin, or gabapentin and one or more other α2/δ ligands, is reduced or eliminated. In this aspect of the invention “α2/δ ligands” include, but are not limited to pregabalin, and to those described in EP641330, WO0128978, WO02/085839, PCT/IB03/00976, EP641330, particularly pregabalin, U.S. Pat. No. 5,563,175, WO9733858, WO9733859, WO9931057, WO9931074, WO9729101, WO02085839, WO9931075, WO9921824, WO0190052, WO0128978, EP0641330, WO9817627, WO0076958, PCT/IB03/00976, EP1178034, EP1201240, WO9931074, WO03000642, WO0222568, WO0230871, WO0230881, WO02100392, WO02100347, WO0242414, WO0232736 and WO0228881, WO03082807 A2; U.S. application Ser. No. 60/507443; U.S. Pat. Nos. 4,024,175, 6,635,673 B1, 6,642,398 B2, and 6,518,289 B1.

In one embodiment, the mutated gene encodes an α2/δ1 polypeptide having an alanine at position 217 (position 241 in the polypeptide having its leader sequence). It is recognized that the corresponding wildtype residue at position 217 is arginine. In another embodiment, the mutated gene encodes an α2/δ1 polypeptide having an alanine at position 215 (position 239 in the polypeptide having its leader sequence). In one embodiment, this mammal is a mouse. While the invention is not limited by any particular mechanism, it is envisioned that this arginine to alanine mutation at positions 217 or 215 (R217A or R215A respectively) or both, and the like, produce a transgenic animal that produces α2/δ1 polypeptides that have reduced or eliminated gabapentin binding activity; while they retain the ability to participate in the formation of functional calcium channels apparently necessary for viability. It is recognized that this mutation can be carried out in a tissue-specific or ubiquitous manner, with respect to the transgenic animal produced to comprise the mutation.

Thus, the invention encompasses a genetically-modified, non-human mammal, wherein the modification results in a mutated α2/δ1 gene wherein the mutation results in an alteration from arginine at position 217 and/or 215 to an alanine at that position. In one embodiment, this mammal exhibits a phenotype in which the binding of gabapentin, or gabapentin and one or more other α2/δ ligands, is reduced or eliminated. In one embodiment,, the mammal is a rodent; in another, a mouse; and in another, the mammal is homozygous for the R217A-like mutation.

In another embodiment, the R217A-like mutation encompasses an arginine to non-arginine substitution in at least one of the two flanking arginines in an arginine-arginine-arginine (RRR) motif. Typically, this RRR motif is the only RRR motif in the entire wildtype α2/δ1. For example, see Genbank accession numbers: M76559(human); U73483; U73484, U73485, U73486, and U73487 (mouse); M86621(rat); and AF077665 (pig). Typically, this RRR motif is found at the tenth to twelfth residues N-terminal to the Von Willebrandt domain (Anantharaman V and Aravind L (2000) Trends Biochem Sci. 25(11):535-7; Bork P and Rohde K (1991) Biochem J. 279 (3): 908-10). For example, in the mouse α2/δ1 polypeptide set forth in genbank accession number U73487, the Von Willebrandt domain is found at residues 227-411 (positions 251435 in the polypeptide having its leader sequence) while the RRR motif is found at residues R215-R217 (positions 239-241 in the polypeptide having its leader sequence). For example, see Genbank references for the α2/δ1 subunit include: M76559(human); U73483, U73484, U73485, U73486, and U73487 (mouse); M86621(rat); and AF077665 (pig).

In another embodiment, the arginine to non-arginine mutation in at least one of the two flanking arginines in an RRR motif is an arginine to an aliphatic amino acid mutation. In another embodiment, this mutation is an arginine to alanine mutation. In another embodiment, this mutation is an arginine to lysine mutation.

In yet another embodiment, the R217A-like mutation encompasses the deletion of at least one of the flanking arginine residues within the RRR motif from the α2/δ1 polypeptide. For example, the invention encompasses deletion of of R239; R241; R239 and R241; or R239, R240 and R241 from the α2/δ1 polypeptide set forth in SEQ ID No. 29. Residues R239-R241 set forth in SEQ ID No. 29 correspond to R215-R217 in the mature polypeptide. By “mature” is intended the same polypeptide, but lacking the leader sequence. In another embodiment, the R217A-like mutation further encompasses the deletion of up to 9 residues immediately N-terminal to the RRR residues, and/or up to 5 residues immediately C-terminal to the RRR residues; in addition to deletion of at least one of the flanking arginine residues within the RRR motif, from the α2/δ1 polypeptide. That is, the R217A-like mutation encompasses the deletion of up to 17 contiguous residues from the α2/δ1 polypeptide, wherein the tenth to twelfth deleted residues consist of the RRR residues described herein; and wherein the deletion comprises at least one of the flanking arginines in the RRR motif. For example, the R217A-like mutation encompasses deleting residues 230 PNKIDLYDVRRRPWYIQ 246 from the mouse α2/δ1 polypeptide set forth in SEQ ID No. 29.

The R217-like mutation of the invention is readily applicable to each of the α2/δ1 subunits set forth in the Genbank references stated herein.

The invention further encompasses a viable, genetically-modified, non-human mammal comprising an α2/δ1 polypeptide comprising an at least one conservative amino acid substitution in addition to one or more R217-like mutations; wherein said conservative amino acid substitution does not significantly alter the biological function of the α2/δ1 polypeptide comprising one or more R217-like mutations. That is, a genetically modified animal or animal cell comprising said R217-like mutation(s) and said conservative amino acid substitution(s)s, is viable and exhibits a phenotype in which the binding of gabapentin, or gabapentin and one or more other α2/δ ligands, is reduced or eliminated.

Conservative amino acid substitutions are well known in the art. For example, typically seen as conservative substitutions are the replacements, one for another, among the aliphatic amino acids Ala, Val, Leu, and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe, Tyr. Guidance concerning which amino acid changes are likely to be phenotypically silent are found in Bowie et al., Science 247:1306-1310 (1990). Conservative amino acid substitutions involve exchanging a member within one group for another member within the same group, including the aromatic, hydrophobic, polar, basic, acidic, and small groups as follows:
Aromatic: Phenylalanine, Tryptophan, Tyrosine;
Hydrophobic: Leucine, Isoleucine, Valine;
Polar: Glutamine, Asparagine
BasiC: Arginine, Lysine, Histidine
Acidic: Aspartic Acid; Glutamic Acid
Small: Alanine, Serine, Threonine, Methionine, Glycine

In a particular embodiment, the invention encompasses a genetically-modified, non-human mammal, wherein the modification results in a mutated α2/δ1 gene encoding a polypeptide selected from the group consisting of:
a) An α2/δ1 polypeptide comprising an arginine to non-arginine substitution in at least one of the two flanking arginines in an RRR motif unique to said polypeptide;
b) An α2/δ1 polypeptide comprising an arginine to aliphatic amino acid substitution in at least one of the two flanking arginines in an RRR motif unique to said polypeptide;
c) An α2/δ1 polypeptide comprising an arginine to alanine substitution in at least one of the two flanking arginines in an RRR motif unique to said polypeptide;
d) An α2/δ1 polypeptide comprising a deletion of at least one of the flanking arginines in an RRR motif unique to said polypeptide;
e) An oα2/δ1 polypeptide comprising
a deletion of up to 9 residues immediately N-terminal to an RRR motif unique to said polypeptide,
a deletion of up to 5 residues immediately C-terminal to an RRR motif unique, and
a deletion of at least one of the flanking arginines in an RRR motif unique to said polypeptide;
f) An α2/δ1 polypeptide comprising
a deletion of up to of up to 9 residues immediately N-terminal to an RRR motif unique to said polypeptide, and
a deletion of at least one of the flanking arginines in an RRR motif unique to said polypeptide;
g) An α2/δ1 polypeptide comprising
a deletion of up to 5 residues immediately C-terminal to an RRR motif unique, and
a deletion of at least one of the flanking arginines in an RRR motif unique to said polypeptide; and
h) An α2/δ1 polypeptide according to A)-g) having at least one conservative amino acid substitution at a position other than a flanking arginines in said RRR motif. In another embodiment, the invention encompasses a genetically-modified animal cell, wherein the modification comprises a mutated α2/δ1 gene. In one embodiment, the cell is an embryonic stem (ES) cell or an ES-like cell; in another, the cell is murine or human; and in another, the cell is homozygous for the modification.

In yet another embodiment, the cell is isolated from a genetically-modified, non-human mammal comprising a modification that results in a mutated α2/δ1 gene. In a particular embodiment, the cell is an embryonic fibroblast, stem cell, neuron, skeletal or cardiac muscle cell, myoblast, brown or white adipocyte, hepatocyte, or pancreatic P cell.

The invention also encompasses a method of identifying a therapeutic agent for epilepsy, neuropathic pain, or anxiety, comprising administering an agent to a genetically-modified mammal of the invention described herein and analyzing the response of the mammal to said agent. In one embodiment this mammal is a non-human mammal homozygous for a mutated α2/δ1 gene. In a particular embodiment, this non-human mammal is a mouse. Methods for assessing epilepsy, neuropathic pain, or anxiety are known in the art and/or otherwise described herein by way of example.

The invention also features a method of identifying a gene that demonstrates modified expression as a result of reduced α2/δ1 activity in an animal cell comprising assessing the expression profile of an animal cell containing a mutated α2/δ1 gene, and comparing the profile to that from a wildtype cell. In one embodiment, the cell is homozygous for the mutated α2/δ1 gene.

In another embodiment, the invention encompasses a method of identifying a protein that demonstrates modified expression or post-translational modification as a result of reduced α2/δ1 activity in an animal cell comprising assessing the proteomic profile of an animal cell containing a mutated α2/δ1gene, and comparing the profile to that from a wildtype cell. In one embodiment, the cell is homozygous for the mutation.

It is recognized that the mammal or the cell of the invention containing the mutations described herein can contain said mutations in an endogenous α2/δ1gene with respect to said mammal or cell, or the gene containing the mutation can be partially or fully heterologous with respect to said mammal or cell. Thus, in this aspect of the invention, the mammal or cell contains an α2/δ1 gene that encodes a polypeptide that is partially or fully heterologous to the mammal or cell and contains an R217-like mutation. For example, the invention encompasses a mouse containing an R217-like mutation as described herein, wherein said R217-like mutation is contained in a non-mouse α2/δ1 mammalian gene or portion thereof.

Alternatively, the mammal or cell contains an α2/δ1 gene that encodes a polypeptide that is endogenous to the mammal or cell but contains an R217-like mutation.

A non-human mammal or an animal cell that is “genetically-modified” is heterozygous or homozygous for a modification that is introduced into the non-human mammal or animal cell, or into a progenitor non-human mammal or animal cell, by genetic engineering. The standard methods of genetic engineering that are available for introducing the modification include homologous recombination, viral vector gene trapping, irradiation, chemical mutagenesis, and the transgenic expression of a nucleotide sequence encoding antisense RNA alone or in combination with catalytic ribozymes. Preferred methods for genetic modification to disrupt a gene are those which modify an endogenous gene by inserting a “foreign nucleic acid sequence” into the gene locus, e.g., by homologous recombination or viral vector gene trapping. A “foreign nucleic acid sequence” is an exogenous sequence that is non-naturally occurring in the gene. This insertion of foreign DNA can occur within any region of the α2/δ1 gene, e.g., in an enhancer, promoter, regulator region, noncoding region, coding region, intron, or exon. The most preferred method of genetic engineering for gene disruption is homologous recombination, in which the foreign nucleic acid sequence is inserted in a targeted manner either alone or in combination with a deletion of a portion of the endogenous gene sequence.

By an α2/δ1 gene that is “mutated” is meant a α2/δ1 gene that is genetically modified such that the cellular activity of the α2/δ1 polypeptide encoded by the mutated gene is altered, reduced, or eliminated in cells that normally express a wildtype version of the α2/δ1 gene (e.g. an R217-like mutation discussed above). When the genetic modification effectively eliminates all wildtype copies of the α2/δ1 gene in a cell (e.g., the genetically-modified, non-human mammal or animal cell is homozygous for the α2/δ1 gene disruption or the only wildtype copy of the α2/δ1 gene originally present is now mutated), the genetic modification results in an alteration, reduction or elimination in α2/δ1 polypeptide activity as compared to a control cell that expresses the wildtype α2/δ1 gene. Preferably, this modification results in a reduction or elimination in α2/δ1 polypeptide activity as compared to a control cell that expresses the wildtype α2/δ1 gene. While the invention is not bound by any particular mechanism, this elimination, reduction, or alteration of α2/δ1 polypeptide activity results because the mutated α2/δ1 gene encodes a mutated polypeptide with altered, e.g., reduced, or eliminated function as compared to a wildtype α2/δ1 polypeptide. In this aspect, the reduction in activity is in any amount that is detectable by comparing the activity of the α2/δ1 polypeptide in the genetically-modified, non-human mammal or animal cell expressing the mutated polypeptide, to that of the corresponding wildtype. In one embodiment, the activity of α2/δ1 polypeptide in the genetically-modified, non-human mammal or animal cell is reduced to 50% or less of wildtype levels; in another, to 25% or less; and in another, to 10% or less of wildtype levels. In a particular embodiment, the α2/δ1 gene mutation results in non-detectable α2/δ1 activity relative to the wildtype.

In one embodiment, the activity referred to in the preceding paragraph is ligand binding activity. Methods for measuring ligand binding to proteins, such as binding assays, are known in the art. In one embodiment, the activity referred to in the preceding paragraph is gabapentin binding activity. Methods for measuring gabapentin binding are known the art, or otherwise disclosed herein. For example, see Wang, M; Offord, J; Oxender D L; and SU T-Z (1999), Biochem. J. 342: 313-320). It is recognized that the binding of other α2/δ ligands can be measured by binding assays.

By a “genetically-modified, non-human mammal containing a mutated α2/δ1 gene” is meant a non-human mammal created by genetic engineering to contain a mutated α2/δ1 gene, as well as a progeny of such non-human mammal that inherits the mutated α2/δ1 gene. A genetically-modified non-human mammal may be produced, for example, by creating a blastocyst or embryo carrying the desired genetic modification and then implanting the blastocyst or embryo in a foster mother for in utero development. The genetically-modified blastocyst or embryo can be made, in the case of mice, by implanting a genetically-modified embryonic stem (ES) cell into a mouse blastocyst or by aggregating ES cells with tetraploid embryos. Alternatively, various species of genetically-modified embryos can be obtained by nuclear transfer. In the case of nuclear transfer, the donor cell is a somatic cell or a pluripotent stem cell, and it is engineered to contain the desired genetic modification that disrupts the α2/δ1 gene. The nucleus of this cell is then transferred into a fertilized or parthenogenetic oocyte that is enucleated; the resultant embryo is reconstituted and developed into a blastocyst. A genetically-modified blastocyst produced by either of the above methods is then implanted into a foster mother according to standard methods well known to those skilled in the art. A “genetically-modified, non-human mammal” includes all progeny of the non-human mammals created by the methods described above, provided that the progeny inherit at least one copy of the genetic modification that disrupts the α2/δ1 gene. It is preferred that all somatic cells and germline cells of the genetically-modified non-human mammal contain the modification. Preferred non-human mammals that are genetically-modified to contain a mutated α2/δ1 gene include rodents, such as mice and rats, cats, dogs, rabbits, guinea pigs, hamsters, sheep, pigs, and ferrets.

By a “genetically-modified animal cell containing a mutated α2/δ1gene” is meant an animal cell, including a human cell, created by genetic engineering to contain a mutated α2/δ1 gene, as well as daughter cells that inherit the mutated α2/δ1 gene. These cells may be genetically-modified in culture according to any standard method known in the art. As an alternative to genetically modifying the cells in culture, non-human mammalian cells may also be isolated from a genetically-modified, non-human mammal that contains a α2/δ1 gene disruption. The animal cells of the invention may be obtained from primary cell or tissue preparations as well as culture-adapted, tumorigenic, or transformed cell lines. These cells and cell lines are derived, for example, from endothelial cells, epithelial cells, islets, neurons and other neural tissue-derived cells, mesothelial cells, osteocytes, lymphocytes, chondrocytes, hematopoietic cells, immune cells, cells of the major glands or organs (e.g., testicle, liver, lung, heart, stomach, pancreas, kidney, and skin), muscle cells (including cells from skeletal muscle, smooth muscle, and cardiac muscle), exocrine or endocrine cells, fibroblasts, and embryonic and other totipotent or pluripotent stem cells (e.g., ES cells, ES-like cells, and embryonic germline (EG) cells, and other stem cells, such as progenitor cells and tissue-derived stem cells). The preferred genetically-modified cells are ES cells, more preferably, mouse or rat ES cells, and, most preferably, human ES cells.

By “reduced α2/δ1 activity” is meant a decrease in the α2/δ ligand binding activity of the α2/δ1 protein as a result of genetic manipulation of the α2/δ1 gene that causes a reduction in the level of functional α2/δ1 polypeptide in a cell. In one embodiment, reduced α2/δ1 activity is in reference to reduced gabapentin binding activity, as a result of the R217A mutation.

By an “ES cell” or an “ES-like cell” is meant a pluripotent stem cell derived from an embryo, from a primordial germ cell, or from a teratocarcinoma, that is capable of indefinite self-renewal as well as differentiation into cell types that are representative of all three embryonic germ layers.

Other features and advantages of the invention will be apparent from the following detailed description and from the claims. While the invention is described in connection with specific embodiments, it will be understood that other changes and modifications that may be practiced are also part of this invention and are also within the scope of the appendant claims. This application is intended to cover any equivalents, variations, uses, or adaptations of the invention that follow, in general, the principles of the invention, including departures from the present disclosure that come within known or customary practice within the art, and that are able to be ascertained without undue experimentation. Additional guidance with respect to making and using nucleic acids and polypeptides is found in standard textbooks of molecular biology, protein science, and immunology (see, e.g., Davis et al., Basic Methods in Molecular Biology, Elsevir Sciences Publishing, Inc., New York, N.Y., 1986; Hames et al., Nucleic Acid Hybridization, IL Press, 1985; Molecular Cloning, Sambrook et al., Current Protocols in Molecular Biology, Eds. Ausubel et al., John Wiley and Sons; Current Protocols in Human Genetics, Eds. Dracopoli et al., John Wiley and Sons; Current Protocols in Protein Science, Eds. John E. Coligan et al., John Wiley and Sons; and Current Protocols in Immunology, Eds. John E. Coligan et al., John Wiley and Sons). All publications, including published patent applications and issued patents, mentioned herein are incorporated by reference in their entireties.

### BRIEF DESCRIPTION OF THE DRAWINGS

FIG. 1 depicts a schematic of the α2/δ1 gene targeting vector, the location for homologous recombination of the vector in the endogenous murine α2/δ1 gene, and the polymerase chain reaction (PCR) strategy used to verify gene targeting.

FIG. 2 depicts the Southern blot analysis of ES Cells.

FIG. 3 depicts gabapentin binding in R217A mutant mouse brain membrains in comparison to that of wildtype mice.

FIG. 4 depicts the effects of pregabalin on formalin-induced hyperalgesia in R217A mutant homozygous mice in comparison to that of heterozygous and wildtype mice.

FIG. 5 depicts the effects of pregabalin in the Tail Suspension Test (TST) in R217A homozygous mutant and wildtype mice.

FIG. 6 depicts Western blots of membranes isolated from mutant (R217A) and wildtype mice.

FIG. 7 depicts the development of chronic constriction injury (CCI)-induced punctate allodynia in R217A and wildtype mice.

FIG. 8 depicts the effects of pregabalin, on CCI-induced static allodynia in R217A and wildtype mice.

FIG. 9 depicts the effects of pregabalin in Maximal Electroshock procedure (MES) in R217A and wildtype mice (“KI” denotes knockin, that is, homozygous for the R217A mutation).

### DETAILED DESCRIPTION OF THE INVENTION

Genetically-modified non-human mammals and animal cells containing a disrupted α2/δ1 gene are provided. The genetically-modified, non-human mammals and genetically-modified animal cells, including human cells, of the invention are heterozygous or homozygous for a modification that disrupts the α2/δ1 gene. The animal cells may be derived by genetically engineering cells in culture, or, in the case of non-human mammalian cells, the cells may be isolated from genetically-modified, non-human mammals.

The genetically-modified non-human mammals and animal cells of the invention are useful for further defining the physiological role of α2/δ1 action; for further defining the therapeutic implications associated with the binding of α2/δ1 ligands to their binding site; for further testing the mechanism of action of α2/δ1 ligands in vivo by treating these mutant animals with the ligands and determining if the compounds are still effective, and/or retain the same characteristics; for determining the relative contribution of α2/δ1 polypeptide in mediating the effects of α2/δ lisgands; for profiling compounds to determine α2/δ1 subtype selectivity of compounds; for identifying signal transduction pathways in which α2/δ ligands are involved; for testing the role of α2/δ in disease processes including neuropathic pain; anxiety, depression, schizophrenia, bipolar disease, and other disorders; testing the role of α2/δ in cardiovascular disease processes; and for using these mutant transgenic animals as breeding intermediates by breeding the animals with other animals, preferably with other α2/δ mutant animals; and for determining the role of leucine in affecting α2/δ activity (Brown J P, Dissanayake V U, Briggs A R, Milic M R, Gee N S (1998) Anal Biochem. January 15; 255(2):236-43).

The mammals and animal cells of the invention are further useful for identifying a gene or genes that demonstrates modified expression as a result of reduced α2/δ1 activity in an animal cell, for identifying a protein or proteins that demonstrates modified expression or post-translational modification as a result of reduced α2/δ1 activity in an animal cell, for producing a transgenic animal having a modified response in an α2/δ1-mediated disorder or activity relative to a wildtype animal, for determining whether the physiological effect of a compound on a disorder or activity involves α2/δ1 subunit polypeptide residues that mediate the physiological effect of an α2/δ1 ligand, for determining whether a compound exerts its physiological effect on a disorder or activity through the same α2/δ1 subunit polypeptide residues as does gabapentin, for identifying compounds that exert their physiological effect on a disorder or activity through an α2/δ1 subunit polypeptide, and for determining a role or roles of an α2/δ1 polypeptide in an activity or disorder.

In order to create genetically modified non-human mammals and animal cells of the invention, the α2/δ1 gene locus may be mutated using techniques for genetic modification known in the art, including chemical mutagenesis (Rinchik, Trends in Genetics 7:15-21, 1991, Russell, Environmental & Molecular Mutagenesis 23 (Suppl. 24): 23-29, 1994), irradiation (Russell, supra), transgenic expression of α2/δ1 gene antisense RNA, either alone or in combination with a catalytic RNA ribozyme sequence (Luyckx et al., Proc. Natl. Acad. Sci. 96: 12174-79, 1999; Sokol et al., Transgenic Research 5: 363-71, 1996; Efrat et al., Proc. Natl. Acad. Sci. USA 91: 2051-55, 1994; Larsson et al., Nucleic Acids Research 22: 2242-48, 1994) and, as further discussed below, the disruption of the α2/δ1 gene by the insertion of a foreign nucleic acid sequence into the α2/δ1 gene locus(Wattler et al. BioTechniques 26:1150-1160, 1999). Preferably, the α2/δ1 gene is mutated by insertion of a foreign nucleic acid sequence, more preferably by means of homologous recombination or by the insertion of a viral vector. Even more preferably, the method of α2/δ1 gene disruption is homologous recombination and includes a deletion of a portion of the endogenous α2/δ1 gene sequence.

The integration of the foreign sequence disrupts the α2/δ gene through one or more of the following mechanisms: by interfering with the α2/δ1 gene transcription or translation process (e.g., by interfering with promoter recognition, or by introducing a transcription termination site or a translational stop codon into the α2/δ1 gene); by distorting the α2/δ1 gene coding sequence such that it no longer encodes an α2/δ1 polypeptide with normal function (e.g., by inserting a foreign coding sequence into the α2/δ1 gene coding sequence, by introducing a frameshift mutation or amino acid(s) substitution; or, in the case of a double crossover event, by deleting a portion of the α2/δ1 gene coding sequence that is required for expression of a functional α2/δ1 protein).

To insert a foreign sequence into an cα2/δ1 gene locus in the genome of a cell to create the genetically modified non-human mammals and animal cells of the invention based upon the present description, the foreign DNA sequence is introduced into the cell according to a standard method known in the art such as electroporation, calcium-phosphate precipitation, retroviral infection, microinjection, biolistics, liposome transfection, DEAE-dextran transfection, or transferrinfection (see, e.g., Neumann et al., EMBO J. 1: 841-845, 1982; Potter et al., Proc. Natl. Acad. Sci USA 81: 7161-65, 1984; Chu et al., Nucleic Acids Res. 15:1311-26, 1987; Thomas and Capecchi, Cell 51: 503-12, 1987; Baum et al., Biotechniques 17:1058-62, 1994; Biewenga et al., J. Neuroscience Methods 71: 67-75, 1997; Zhang et al., Biotechniques 15: 868-72, 1993; Ray and Gage, Biotechniques 13: 598-603, 1992; Lo, Mol. Cell. Biol. 3:1803-14, 1983; Nickoloff et al., Mol. Biotech. 10: 93-101, 1998; Linney et al., Dev. Biol. (Orlando) 213: 207-16, 1999; Zimmer and Gruss, Nature 338:150-153, 1989; and Robertson et al., Nature 323: 445-48, 1986). The preferred method for introducing foreign DNA into a cell is electroporation.

1. Homologous Recombination

The method of homologous recombination targets the α2/δ1 gene for disruption by introducing a α2/δ gene targeting vector into a cell containing a α2/δ1 gene. The vector targets the α2/δ1 gene using a nucleotide sequence in the vector that is homologous to the α2/δ1 gene. This homologous region facilitates hybridization between the vector and the endogenous sequence of the α2/δ1 gene. Upon hybridization, the probability of a crossover event between the targeting vector and genomic sequences increases greatly. Such a crossover event results in the integration of the vector sequence into the α2/δ1 gene locus and the likely functional disruption of the α2/δ1 gene.

General principles regarding the construction of vectors used for targeting are reviewed in Bradley et al. (Biotechnol. 10: 534, 1992) and Wattler et al. (BioTechniques 26:1150-1160, 1999). Two types of vectors that may be used to insert DNA by homologous recombination are insertion vectors and replacement vectors. The preferred vectors for preparing the genetically modified non-human mammals and animal cells of the invention by homologous recombination are replacement vectors

Insertion vectors are a circular DNA molecule which include a region of α2/δ gene homology with a double stranded break. Following hybridization between the homology region and the endogenous α2/δ1 gene, a single crossover event at the double stranded break results in the insertion of the entire vector sequence into the endogenous gene at the site of crossover A replacement vector is linear rather than circular. Replacement vector integration into the α2/δ1 gene requires a double crossover event, i.e. crossing over at two sites of hybridization between the targeting vector and the α2/δ gene. This double crossover event results in the integration of a vector sequence that is sandwiched between the two sites of crossover into the α2/δ1 gene and the deletion of the corresponding endogenous α2/δ1 gene sequence that originally spanned between the two sites of crossover (see, for example, Thomas and Capecchi et al., Cell 51: 503-12, 1987; Mansour et al., Nature 336: 348-52, 1988; Mansour et al., Proc. Natl. Acad. Sci. USA 87: 7688-7692, 1990; and Mansour, GATA 7: 219-227, 1990).

A region of homology in a targeting vector used to create the genetically modified non-human mammals and animal cells of the invention is generally at least 100 nucleotides in length. Most preferably, the homology region is at least 1-5 kilobases (kb) in length. Although there is no demonstrated minimum length or minimum degree of relatedness required for a homology region, targeting efficiency for homologous recombination generally corresponds with the length and the degree of relatedness between the targeting vector and the α2/δ1 gene locus. In the case where a replacement vector is used, and a portion of the endogenous α2/δ1 gene is deleted upon homologous recombination, an additional consideration is the size of the deleted portion of the endogenous α2/δ1 gene. If this portion of the endogenous α2/δ1 gene is greater than 1 kb in length, then a targeting cassette with regions of homology that are longer than 1 kb is recommended to enhance the efficiency of recombination. Further guidance regarding the selection and use of sequences effective for homologous recombination, based on the present description, is described in the literature (see, e.g., Deng and Capecchi, Mol. Cell. Biol. 12: 3365-3371, 1992; Bollag et al., Annu. Rev. Genet. 23: 199-225, 1989; and Waldman and Liskay, Mol. Cell. Biol. 8: 5350-5357, 1988).

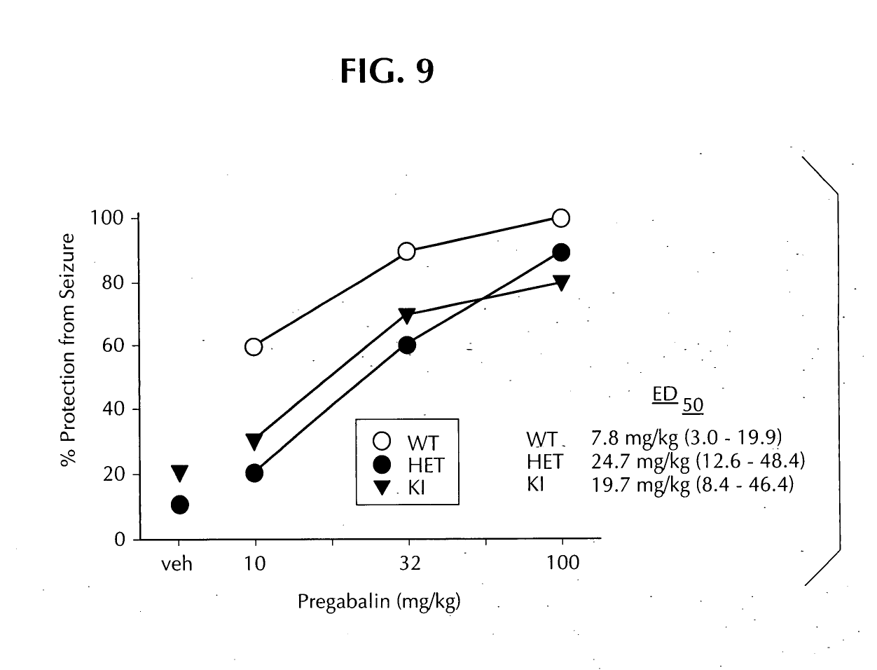

As those skilled in the art will recognize based upon the present invention, a wide variety of cloning vectors may be used as vector backbones in the construction of the α2/δ1 gene targeting vectors of the present invention, including pBluescript-related plasmids (e.g., Bluescript KS+11), pQE70, pQE60, pQE-9, pBS, pD10, phagescript, phiX174, pBK Phagemid, pNH8A, pNH16a, pNH18Z, pNH46A, ptrc99a, pKK223-3, pKK233-3, pDR540, and pRIT5 PWLNEO, pSV2CAT, pXT1, pSG (Stratagene), pSVK3, PBPV, PMSG, and pSVL, pBR322 and pBR322-based vectors, pMB9, pBR325, pKH47, pBR328, pHC79, phage Charon 28, pKB11, pKSV-10, pK19 related plasmids, pUC plasmids, and the pGEM series of plasmids. These vectors are available from a variety of commercial sources (e.g., Boehringer Mannheim Biochemicals, Indianapolis, Ind.; Qiagen, Valencia, Calif.; Stratagene, La Jolla, Calif.; Promega, Madison, Wis.; and New England Biolabs, Beverly, Mass.). However, any other vectors, e.g. plasmids, viruses, or parts thereof, may be used as long as they are replicable and viable in the desired host. The vector may also comprise sequences which enable it to replicate in the host whose genome is to be modified. The use of such a vector can expand the interaction period during which recombination can occur, increasing the efficiency of targeting (see Molecular Biology, ed. Ausubel et al, Unit 9.16, FIG. 9.16.1).

The specific host employed for propagating the targeting vectors of the present invention is not critical. Examples include E. coli K12 RR1 (Bolivar et al., Gene 2: 95, 1977), E. coli K12 HB101 (ATCC No. 33694), E. coli MM21 (ATCC No. 336780), E. coli DH1 (ATCC No. 33849), E. coli strain DH5a, and E. coli STBL2. Alternatively, hosts such as C. cerevisiae or B. subtilis can be used. The above-mentioned hosts are available commercially (e.g., Stratagene, La Jolla, Calif.; and Life Technologies, Rockville, Md.).

In order to create a targeting vector, a α2/δ1 gene targeting construct is added to a vector backbone, such as, for example, a vector backbone described above. The α2/δ1 gene targeting constructs of the invention have at least one α2/δ1 gene homology region. To make the α2/δ1 gene homology regions, a α2/δ1 genomic or cDNA sequence is used as a basis for producing PCR primers. These primers are used to amplify the desired region of the α2/δ1 sequence by high fidelity PCR amplification (Mattila et al., Nucleic Acids Res. 19: 4967, 1991; Eckert and Kunkel 1: 17, 1991; and U.S. Pat. No. 4,683,202). The genomic sequence is obtained from a genomic clone library or from a preparation of genomic DNA, preferably from the animal species that is to be targeted for α2/δ1 gene disruption. A α2/δ1 cDNA sequence can be used in making a α2/δ1 targeting vector (e.g., Genbank U22445 (murine), Genbank M95936 (human), Genbank D30041 (rat), Genbank AF181260 (hen), BG410200 (Xenopus)). Preferably, the targeting constructs of the invention also include an exogenous nucleotide sequence encoding a positive marker protein. The stable expression of a positive marker after vector integration confers an identifiable characteristic on the cell, ideally, without compromising cell viability. Therefore, in the case of a replacement vector, the marker gene is positioned between two flanking homology regions so that it integrates into the α2/δ1 gene following the double crossover event in a manner such that the marker gene is positioned for expression after integration.

It is preferred that the positive marker protein confer a selectable phenotypic characteristic, such as, for example, a characteristic that enhances the survival of the cell under otherwise lethal conditions. Thus, by imposing the selectable condition, one may isolate cells that stably express the positive selectable marker-encoding vector sequence from other cells that have not successfully integrated the vector sequence on the basis of viability. Examples of positive selectable marker proteins (and their agents of selection) include neo (G418 or kanomycin), hyg (hygromycin), hisD (histidinol), gpt (xanthine), ble (bleomycin), and hprt (hypoxanthine) (see, e.g., Capecchi and Thomas, U.S. Pat. No. 5,464,764, and Capecchi, Science 244: 1288-92, 1989). Other positive markers that may also be used as an alternative to a selectable marker include reporter proteins such as β-galactosidase, firefly luciferase, or GFP (see, e.g., Current Protocols in Cytometry, Unit 9.5, and Current Protocols in Molecular Biology, Unit 9.6, John Wiley & Sons, New York, N.Y., 2000).

The above-described positive selection step does not distinguish between cells that have integrated the vector by targeted homologous recombination at the α2/δ1 gene locus versus random, non-homologous integration of vector sequence into any chromosomal position. Therefore, when using a replacement vector for homologous recombination to make the genetically modified non-human mammals and animal cells of the invention, it is also preferred to include a nucleotide sequence encoding a negative selectable marker protein. Expression of a negative selectable marker causes a cell expressing the marker to lose viability when exposed to a certain agent (i.e., the marker protein becomes lethal to the cell under certain selectable conditions). Examples of negative selectable markers (and their agents of lethality) include herpes simplex virus thymidine kinase (gancyclovir or 1,2-deoxy-2-fluoro-α-d-arabinofuransyl-5-iodouracil), Hprt (6-thioguanine or 6-thioxanthine), and diphtheria toxin, ricin toxin, and cytosine deaminase (5-fluorocytosine).

The nucleotide sequence encoding the negative selectable marker is positioned outside of the two homology regions of the replacement vector. Given this positioning, cells will only integrate and stably express the negative selectable marker if integration occurs by random, non-homologous recombination; homologous recombination between the α2/δ1 gene and the two regions of homology in the targeting construct excludes the sequence encoding the negative selectable marker from integration. Thus, by imposing the negative condition, cells that have integrated the targeting vector by random, non-homologous recombination lose viability.

The above-described combination of positive and negative selectable markers is preferred in a targeting construct used to make the genetically modified non-human mammals and animal cells of the invention because a series of positive and negative selection steps can be designed to more efficiently select only those cells that have undergone vector integration by homologous recombination, and, therefore, have a potentially mutated α2/δ1 gene. Further examples of positive-negative selection schemes, selectable markers, and targeting constructs are described, for example, in U.S. Pat. No. 5,464,764, WO 94/06908 (issued in the U.S. as U.S. Pat. No. 5,859,312), and Valancius and Smithies, Mol. Cell. Biol. 11: 1402, 1991.

In order for a marker protein to be stably expressed upon vector integration, the targeting vector may be designed so that the marker coding sequence is operably linked to the endogenous α2/δ1 gene promoter upon vector integration. Expression of the marker is then driven by the α2/δ1 gene promoter in cells that normally express the α2/δ1 gene. Alternatively, each marker in the targeting construct of the vector may contain its own promoter that drives expression independent of the α2/δ1 gene promoter. This latter scheme has the advantage of allowing for expression of markers in cells that do not typically express the α2/δ1 gene (Smith and Berg, Cold Spring Harbor Symp. Quant. Biol. 49: 171, 1984; Sedivy and Sharp, Proc. Natl. Acad. Sci. (USA) 86: 227, 1989; Thomas and Capecchi, Cell 51: 503, 1987).

Exogenous promoters that can be used to drive marker gene expression include cell-specific or stage-specific promoters, constitutive promoters, and inducible or regulatable promoters. Non-limiting examples of these promoters include the herpes simplex thymidine kinase promoter, cytomegalovirus (CMV) promoter/enhancer, SV40 promoters, PGK promoter, PMC1-neo, metallothionein promoter, adenovirus late promoter, vaccinia virus 7.5K promoter, avian beta globin promoter, histone promoters (e.g., mouse histone H3-614), beta actin promoter, neuron-specific enolase, muscle actin promoter, and the cauliflower mosaic virus 35S promoter (see generally, Sambrook et al., Molecular Cloning, Vols. I-III, Cold Spring Harbor Laboratory Press, Cold Spring Harbor, N.Y., 1989, and Current Protocols in Molecular Biology, John Wiley & Sons, New York, N.Y., 2000; Stratagene, La Jolla, Calif.).

To confirm whether cells have integrated the vector sequence into the targeted α2/δ1 gene locus while making the genetically modified non-human mammals and animal cells of the invention, primers or genomic probes that are specific for the desired vector integration event can be used in combination with PCR or Southern blot analysis to identify the presence of the desired vector integration into the α2δ1 gene locus (Erlich et al., Science 252: 1643-51, 1991; Zimmer and Gruss, Nature 338: 150, 1989; Mouellic et al., Proc. Natl. Acad. Sci. (USA) 87: 4712, 1990; and Shesely et al., Proc. Natl. Acad. Sci. (USA) 88: 4294, 1991).

3. Gene Trapping

Another method available for inserting a foreign nucleic acid sequence into the α2/δ1 gene locus to disrupt the α2/δ1 gene, based on the present description, is gene trapping. This method takes advantage of the cellular machinery present in all mammalian cells that splices exons into mRNA to insert a gene trap vector coding sequence into a gene in a random fashion. Once inserted, the gene trap vector creates a mutation that may disrupt the trapped α2/δ1 gene. In contrast to homologous recombination, this system for mutagenesis creates largely random mutations. Thus, to obtain a genetically-modified cell that contains a mutated α2/δ1 gene, cells containing this particular mutation must be identified and selected from a pool of cells that contain random mutations in a variety of genes.

Gene trapping systems and vectors have been described for use in genetically modifying murine cells and other cell types (see, e.g., Allen et al., Nature 333: 852-55, 1988; Bellen et al., Genes Dev. 3: 1288-1300, 1989; Bier et al., Genes Dev. 3: 1273-1287, 1989; Bonnerot et al., J. Virol. 66: 4982-91, 1992; Brenner et al., Proc. Nat. Acad. Sci. USA 86: 5517-21, 1989; Chang et al., Virology 193: 737-47, 1993; Friedrich and Soriano, Methods Enzymol. 225: 681-701, 1993; Friedrich and Soriano, Genes Dev. 5: 1513-23, 1991; Goff, Methods Enzymol. 152: 469-81, 1987; Gossler et al., Science 244: 463-65, 1989; Hope, Develop. 113: 399-408, 1991; Kerr et al., Cold Spring Harb. Symp. Quant. Biol. 2: 767-776, 1989; Reddy et al., J. Virol. 65: 1507-1515, 1991; Reddy et al., Proc. Natl. Acad. Sci. U.S.A. 89: 6721-25, 1992; Skarnes et al., Genes Dev. 6: 903-918, 1992; von Melchner and Ruley, J. Virol. 63: 3227-3233, 1989; and Yoshida et al., Transgen. Res. 4: 277-87, 1995).

Promoter trap vectors (or 5′ vectors) contain, in 5′ to 3′ order, a splice acceptor sequence followed by an exon, which is typically characterized by a translation initiation codon and open reading frame and/or an internal ribosome entry site. In general, these promoter trap vectors do not contain promoters or operably linked splice donor sequences. Consequently, after integration into the cellular genome of the host cell, the promoter trap vector sequence intercepts the normal splicing of the upstream gene and acts as a terminal exon. Expression of the vector coding sequence is dependent upon the vector integrating into an intron of the mutated gene in the proper reading frame. In such a case, the cellular splicing machinery splices exons from the trapped gene upstream of the vector coding sequence (Zambrowicz et al., WO 99/50426 and U.S. Pat. No. 6,080,576). An alternative method for producing an effect similar to the above-described promoter trap vector is a vector that incorporates a nested set of stop codons present in, or otherwise engineered into, the region between the splice acceptor of the promoter trap vector and the translation initiation codon or polyadenylation sequence. The coding sequence can also be engineered to contain an independent ribosome entry site (IRES) so that the coding sequence will be expressed in a manner largely independent of the site of integration within the host cell genome. Typically, but not necessarily, an IRES is used in conjunction with a nested set of stop codons.

Another type of gene trapping scheme uses a 3′ gene trap vector. This type of vector contains, in operative combination, a promoter region, which mediates expression of an adjoining coding sequence, the coding sequence, and a splice donor sequence that defines the 3′ end of the coding sequence exon. After integration into a host cell genome, the transcript expressed by the vector promoter is spliced to a splice acceptor sequence from the trapped gene that is located downstream of the integrated gene trap vector sequence. Thus, the integration of the vector results in the expression of a fusion transcript comprising the coding sequence of the 3′ gene trap cassette and any downstream cellular exons, including the terminal exon and its polyadenylation signal. When such vectors integrate into a gene, the cellular splicing machinery splices the vector coding sequence upstream of the 3′ exons of the trapped gene. One advantage of such vectors is that the expression of the 3′ gene trap vectors is driven by a promoter within the gene trap cassette and does not require integration into a gene that is normally expressed in the host cell (Zambrowicz et al., WO 99/50426 and U.S. Pat. No. 6,080,576). Examples of transcriptional promoters and enhancers that may be incorporated into the 3′ gene trap vector include those discussed above with respect to targeting vectors.

The viral vector backbone used as the structural component for the promoter or 3′ gene trap vector may be selected from a wide range of vectors that can be inserted into the genome of a target cell. Suitable backbone vectors include, but are not limited to, herpes simplex virus vectors, adenovirus vectors, adeno-associated virus vectors, retroviral vectors, lentiviral vectors, pseudorabies virus, alpha-herpes virus vectors, and the like. A thorough review of viral vectors, in particular, viral vectors suitable for modifying nonreplicating cells and how to use such vectors in conjunction with the expression of an exogenous polynucleotide sequence, can be found in Viral Vectors: Gene Therapy and Neuroscience Applications, Eds. Caplitt and Loewy, Academic Press, San Diego, 1995.

Preferably, retroviral vectors are used for gene trapping. These vectors can be used in conjunction with retroviral packaging cell lines such as those described in U.S. Pat. No. 5,449,614. Where non-murine mammalian cells are used as target cells for genetic modification, amphotropic or pantropic packaging cell lines can be used to package suitable vectors (Ory et al., Proc. Natl. Acad. Sci., USA 93: 11400-11406, 1996). Representative retroviral vectors that can be adapted to create the presently described 3′ gene trap vectors are described, for example, in U.S. Pat. No. 5,521,076.

The gene trapping vectors may contain one or more of the positive marker genes discussed above with respect to targeting vectors used for homologous recombination. Similar to their use in targeting vectors, these positive markers are used in gene trapping vectors to identify and select cells that have integrated the vector into the cell genome. The marker gene may be engineered to contain an independent ribosome entry site (IRES) so that the marker will be expressed in a manner largely independent of the location in which the vector has integrated into the target cell genome.

Given that gene trap vectors will integrate into the genome of infected host cells in a fairly random manner, a genetically-modified cell having a mutated α2/δ1 gene must be identified from a population of cells that have undergone random vector integration. Preferably, the genetic modifications in the population of cells are of sufficient randomness and frequency such that the population represents mutations in essentially every gene found in the cell's genome, making it likely that a cell with a mutated α2/δ1 gene will be identified from the population (see Zambrowicz et al., WO 99/50426; Sands et al., WO 98/14614 and U.S. Pat. No. 6,080,576).

Individual mutant cell lines containing a mutated α2/δ1 gene are identified in a population of mutated cells using, for example, reverse transcription and polymerase chain reaction (PCR) to identify a mutation in a α2/δ1 gene sequence. This process can be streamlined by pooling clones. For example, to find an individual clone containing a mutated α2/δ1 gene, RT-PCR is performed using one primer anchored in the gene trap vector and the other primer located in the α2/δ1 gene sequence. A positive RT-PCR result indicates that the vector sequence is encoded in the (α2/δ1 gene transcript, indicating that the α2/δ1 gene has been mutated by a gene trap integration event (see, e.g., Sands et al., WO 98/14614, U.S. Pat. No. 6,080,576).

4. Temporal, Spatial, and Inducible α2/δ1 Gene Disruptions

In certain embodiments of the present invention, a functional disruption of the endogenous α2/δ1 gene occurs at specific developmental or cell cycle stages (temporal disruption) or in specific cell types (spatial disruption). In other embodiments, the α2/δ1 gene disruption is inducible when certain conditions are present. A recombinase excision system, such as a Cre-Lox system, may be used to activate or inactivate the α2/δ1 gene at a specific developmental stage, in a particular tissue or cell type, or under particular environmental conditions. Generally, methods utilizing Cre-Lox technology are carried out as described by Torres and Kuhn, Laboratory Protocols for Conditional Gene Targeting, Oxford University Press, 1997. Methodology similar to that described for the Cre-Lox system can also be employed utilizing the FLP-FRT system. Further guidance regarding the use of recombinase excision systems for conditionally disrupting genes by homologous recombination or viral insertion is provided, for example, in U.S. Pat. No. 5,626,159, U.S. Pat. No. 5,527,695, U.S. Pat. No. 5,434,066, WO 98/29533, U.S. Pat. No. 6,228,639, Orban et al., Proc. Nat. Acad. Sci. USA 89: 6861-65, 1992; O'Gorman et al., Science 251:1351-55, 1991; Sauer et al., Nucleic Acids Research 17: 147-61, 1989; Barinaga, Science 265: 26-28, 1994; and Akagi et al., Nucleic Acids Res. 25: 1766-73, 1997. More than one recombinase system can be used to genetically modify a non-human mammal or animal cell of the present invention.

When using homologous recombination to disrupt the α2/δ1 gene in a temporal, spatial, or inducible fashion, using a recombinase system such as the Cre-Lox system, a portion of the α2/δ1 gene coding region is replaced by a targeting construct comprising the α2/δ1 gene coding region flanked by loxP sites. Non-human mammals and animal cells carrying this genetic modification contain a functional, loxP-flanked α2/δ1 gene. The temporal, spatial, or inducible aspect of the α2/δ1 gene disruption is caused by the expression pattern of an additional transgene, a Cre recombinase transgene, that is expressed in the non-human mammal or animal cell under the control of the desired spatially-regulated, temporally-regulated, or inducible promoter, respectively. A Cre recombinase targets the loxP sites for recombination. Therefore, when Cre expression is activated, the LoxP sites undergo recombination to excise the sandwiched α2/δ1 gene coding sequence, resulting in a functional disruption of the α2/δ1 gene (Rajewski et al., J. Clin. Invest. 98: 600-03, 1996; St.-Onge et al., Nucleic Acids Res. 24: 3875-77, 1996; Agah et al., J. Clin. Invest. 100: 169-79, 1997; Brocard et al., Proc. Natl. Acad. Sci. USA 94: 14559-63, 1997; Feil et al., Proc. Natl. Acad. Sci. USA 93: 10887-90, 1996; and Kuihn et al., Science 269:1427-29, 1995). A cell containing both a Cre recombinase transgene and loxP-flanked α2/δ1 gene can be generated through standard transgenic techniques or, in the case of genetically-modified, non-human mammals, by crossing genetically-modified, non-human mammals wherein one parent contains a loxP flanked α2/δ1 gene and the other contains a Cre recombinase transgene under the control of the desired promoter. Further guidance regarding the use of recombinase systems and specific promoters to temporally, spatially, or conditionally disrupt the α2/δ1 gene is found, for example, in Sauer, Meth. Enz. 225: 890-900, 1993, Gu et al., Science 265: 103-06, 1994, Araki et al., J. Biochem. 122: 977-82, 1997, Dymecki, Proc. Natl. Acad. Sci. 93: 6191-96, 1996, and Meyers et al., Nature Genetics 18: 136-41, 1998.

An inducible disruption of the α2/δ1 gene can also be achieved by using a tetracycline responsive binary system (Gossen and Bujard, Proc. Natl. Acad. Sci. USA 89: 5547-51, 1992). This system involves genetically modifying a cell to introduce a Tet promoter into the endogenous α2/δ1 gene regulatory element and a transgene expressing a tetracycline-controllable repressor (TetR). In such a cell, the administration of tetracycline activates the TetR which, in turn, inhibits α2/δ1 gene expression and, therefore, disrupts the α2/δ1 gene (St.-Onge et al., Nucleic Acids Res. 24: 3875-77, 1996, U.S. Pat. No. 5,922,927).

The above-described systems for temporal, spatial, and inducible disruptions of the α2/δ1 gene can also be adopted when using gene trapping as the method of genetic modification, for example, as described, in WO 98/29533 and U.S. Pat. No. 6,288,639, for creating the genetically modified non-human mammals and animal cells of the invention.

5. Preparation of Genetically-Modified Animal Cells

The above-described methods for genetic modification can be used to disrupt a α2/δ1 gene in virtually any type of somatic or stem cell derived from an animal to create the genetically modified animal cells of the invention. Genetically-modified animal cells of the invention include, but are not limited to, mammalian cells, including human cells, and avian cells. These cells may be derived from genetically engineering any animal cell line, such as culture-adapted, tumorigenic, or transformed cell lines, or they may be isolated from a genetically-modified, non-human mammal carrying the desired α2/δ1 genetic modification. The cells may be heterozygous or homozygous for the mutated α2/δ1 gene. To obtain cells that are homozygous for the α2/δ1 gene disruption (−/−), direct, sequential targeting of both alleles can be performed. This process can be facilitated by recycling a positive selectable marker. According to this scheme the nucleotide sequence encoding the positive selectable marker is removed following the disruption of one allele using the Cre-Lox P system. Thus, the same vector can be used in a subsequent round of targeting to disrupt the second α2/δ1 gene allele (Abuin and Bradley, Mol. Cell. Biol. 16:1851-56, 1996; Sedivy et al., T.I.G. 15: 88-90, 1999; Cruz et al., Proc. Natl. Acad. Sci. (USA) 88: 7170-74, 1991; Mortensen et al., Proc. Natl. Acad. Sci. (USA) 88: 7036-40, 1991; te Riele et al., Nature (London) 348: 649-651, 1990).

An alternative strategy for obtaining ES cells that are α2/δ1 is the homogenotization of cells from a population of cells that is heterozygous for the α2/δ1 gene disruption (α2/δ1 +/−). The method uses a scheme in which α2/δ1+/− targeted clones that express a selectable drug resistance marker are selected against a very high drug concentration; this selection favors cells that express two copies of the sequence encoding the drug resistance marker and are, therefore, homozygous for the α2/δ1 gene disruption (Mortensen et al., Mol. Cell. Biol. 12: 2391-95, 1992). In addition, genetically-modified animal cells can be obtained from genetically-modified α2/δ1−/− non-human mammals that are created by mating non-human mammals that are α2/δ1+/− in germline cells, as further discussed below.

Following the genetic modification of the desired cell or cell line, the α2/δ1 gene locus can be confirmed as the site of modification by PCR analysis according to standard PCR or Southern blotting methods known in the art (see, e.g., U.S. Pat. No. 4,683,202; and Erlich et al., Science 252:1643, 1991). Further verification of the functional disruption of the α2/δ1 gene may also be made if α2/δ1 gene messenger RNA (mRNA) levels and/or α2/δ1 polypeptide levels are reduced in cells that normally express the α2/δ1 gene. Measures of α2/δ1 gene mRNA levels may be obtained by using reverse transcriptase mediated polymerase chain reaction (RT-PCR), Northern blot analysis, or in situ hybridization. The quantification of α2/δ1 polypeptide levels produced by the cells can be made, for example, by standard immunoassay methods known in the art. Such immunoassays include, but are not limited to, competitive and non-competitive assay systems using techniques such as RIAs (radioimmunoassays), ELISAs (enzyme-linked immunosorbent assays), “sandwich” immunoassays, immunoradiometric assays, gel diffusion precipitin reactions, immunodiffusion assays, in situ immunoassays (using, for example, colloidal gold, enzymatic, or radioisotope labels), Western blots, 2-dimensional gel analysis, precipitation reactions, immunofluorescence assays, protein A assays, and immunoelectrophoresis assays.

Preferred genetically-modified animal cells of the invention are embryonic stem (ES) cells and ES-like cells. These cells are derived from the preimplantation embryos and blastocysts of various species, such as mice (Evans et al., Nature 129: 154-156, 1981; Martin, Proc. Natl. Acad. Sci., USA, 78: 7634-7638, 1981), pigs and sheep (Notanianni et al., J. Reprod. Fert. Suppl., 43: 255-260, 1991; Campbell et al., Nature 380: 64-68,1996) and primates, including humans (Thomson et al., U.S. Pat. No. 5,843,780, Thomson et al., Science 282: 1145-1147, 1995; and Thomson et al., Proc. Natl. Acad. Sci. USA 92: 7844-7848, 1995).

These types of cells are pluripotent, that is, under proper conditions, they differentiate into a wide variety of cell types derived from all three embryonic germ layers: ectoderm, mesoderm and endoderm. Depending upon the culture conditions, a sample of ES cells can be cultured indefinitely as stem cells, allowed to differentiate into a wide variety of different cell types within a single sample, or directed to differentiate into a specific cell type, such as macrophage-like cells, neuronal cells, cardiomyocytes, chondrocytes, adipocytes, smooth muscle cells, endothelial cells, skeletal muscle cells, keratinocytes, and hematopoietic cells, such as eosinophils, mast cells, erythroid progenitor cells, or megakaryocytes. Directed differentiation is accomplished by including specific growth factors or matrix components in the culture conditions, as further described, for example, in Keller et al., Curr. Opin. Cell Biol. 7: 862-69, 1995, Li et al., Curr. Biol. 8: 971, 1998, Klug et al., J. Clin. Invest. 98: 216-24, 1996, Lieschke et al., Exp. Hematol. 23: 328-34, 1995, Yamane et al., Blood 90: 3516-23, 1997, and Hirashima et al., Blood 93: 1253-63, 1999.

The particular ES cell line that is used for genetic modification is not critical. For example, for those embodiments of this invention wherein murine ES cell lines are used, such cells may include AB-1 (McMahon and Bradley, Cell 62: 1073-85, 1990), E14 (Hooper et al., Nature 326: 292-95, 1987), D3 (Doetschman et al., J. Embryol. Exp. Morph. 87: 27-45, 1985), CCE (Robertson et al, Nature 323: 445-48, 1986), RW4 (Genome Systems, St. Louis, Mo.), and DBA/llacJ (Roach et al., Exp. Cell Res. 221: 520-25, 1995).

6. Preparation of Genetically-Modified, Non-human Mammals

The genetically-modified animal cells of this invention may be used to prepare genetically-modified, non-human mammals of this invention. In one embodiment, genetically-modified ES cells of this invention may be used to generate genetically-modified non-human mammals, according to published procedures (Robertson, 1987, Teratocarcinomas and Embryonic Stem Cells: A Practical Approach, Ed. E. J. Robertson, Oxford: IRL Press, pp. 71-112, 1987; Zjilstra et al., Nature 342: 435-438, 1989; and Schwartzberg et al., Science 246: 799-803, 1989).

In a preferred embodiment, said ES cells are murine ES cells (e.g. 129S6/SvEV) and said genetically-modified non-human mammals, are mice. For example, in the preparation of such genetically-modified mice, murine ES cells containing the desired functional disruption of the α2/δ1 gene are used. The murine ES cells to be used are first confirmed to contain the desired functional disruption of the α2/δ1 gene, as described above.

The α2/δ1 mutated ES cells may then be used to generate chimeric mice according to methods known in the art (Capecchi, Trends Genet. 5: 70, 1989). For example, the α2/δ1 mutated ES cells may be injected into suitable blastocyst hosts. The particular mouse blastocysts employed in the practice of the present invention are not critical. Examples of such blastocysts will be known to those with skill in the art in light of the present description, and include blastocysts derived from C57BL6 mice, C57BL6 Albino mice, Swiss outbred mice, CFLP mice, and MFI mice. Alternatively ES cells may be sandwiched between tetraploid embryos in aggregation wells (Nagy et al., Proc. Natl. Acad. Sci. USA 90: 8424-8428, 1993).

The blastocysts or embryos containing the genetically-modified ES cells are then implanted in pseudopregnant female mice and allowed to develop in utero (Hogan et al., Manipulating the Mouse Embryo: A Laboratory Manual, Cold Spring Harbor Laboratory press, Cold Spring Harbor, N.Y. 1988; and Teratocarcinomas and Embryonic Stem Cells: A Practical Approach, E. J. Robertson, ed., IRL Press, Washington, D.C., 1987). The offspring born to the foster mothers may be screened to identify those that are chimeric for the □\overline{□} □₁ gene disruption. Generally, such offspring contain some cells that are derived from the genetically-modified donor ES cell as well as other cells derived from the original blastocyst. In such circumstances, offspring may be screened initially for mosaic coat color, where a coat color selection strategy has been employed, to distinguish cells derived from the donor ES cell from the other cells of the blastocyst. Alternatively, DNA from tail tissue of the offspring can be used to identify mice containing the genetically-modified cells. The mating of chimeric mice that contain the α2/δ1 gene disruption in germ line cells produces progeny that possess the α2/δ1 gene disruption in all germ line cells and somatic cells. Mice that are heterozygous for the α2/δ1 gene disruption can then be crossed to produce homozygotes (see, e.g., U.S. Pat. No. 5,557,032, and U.S. Pat. No. 5,532,158).

An alternative to the above-described ES cell technology for transferring a genetic modification from a cell to a whole animal is to use nuclear transfer. This method can be employed to make other genetically-modified, non-human mammals besides mice, for example, sheep (McCreath et al., Nature 29: 1066-69, 2000; Campbell et al., Nature 389: 64-66, 1996; and Schnieke et al., Science 278: 2130-33, 1997) and calves (Cibelli et al., Science 280: 1256-58, 1998). Briefly, somatic cells (e.g., fibroblasts) or pluripotent stem cells (e.g., ES-like cells) are selected as nuclear donors and are genetically-modified to contain a functional disruption of the α2/δ1 gene. When inserting a DNA vector into a somatic cell to mutate the α2/δ1 gene, it is preferred that a promoterless marker be used in the vector such that vector integration into the α2/δ1 gene results in expression of the marker under the control of the α2/δ1 gene promoter (Sedivy and Dutriaux, T.I.G. 15: 88-90, 1999; McCreath et al., Nature 29: 1066-69, 2000). Nuclei from donor cells which have the appropriate α2/δ1 gene disruption are then transferred to fertilized or parthenogenetic oocytes that are enucleated (Campbell et al., Nature 380: 64, 1996; Wilmut et al., Nature 385: 810, 1997). Embryos are reconstructed, cultured to develop into the morula/blastocyst stage, and transferred into foster mothers for full term in utero development.

The present invention also encompasses the progeny of the genetically-modified, non-human mammals and genetically-modified animal cells. While the progeny are heterozygous or homozygous for the genetic modification that disrupts the α2/δ1 gene, they may not be genetically identical to the parent non-human mammals and animal cells due to mutations or environmental influences, besides that of the original genetic disruption of the α2/δ1 gene, that may occur in succeeding generations.

The cells from a non-human genetically modified animal can be isolated from tissue or organs using techniques known to those of skill in the art. In one embodiment, the genetically modified cells of the invention are immortalized. In accordance with this embodiment, cells can be immortalized by genetically engineering the telomerase gene, an oncogene, e.g., mos or v-src, or an apoptosis-inhibiting gene, e.g., bcl-2, into the cells. Alternatively, cells can be immortalized by fusion with a hybridization partner utilizing techniques known to one of skill in the art.

7. “Humanized” Non-human Mammals and Animal Cells

The genetically-modified non-human mammals and animal cells (non-human) of the invention containing a mutated endogenous α2/δ1 gene can be further modified to express the human aα2/δ1 sequence (referred to herein as “humanized”). A preferred method for humanizing cells involves replacing the endogenous α2/δ1 sequence with nucleic acid sequence encoding the human α2/δ1 sequence (Jakobsson et al., Proc. Natl. Acad. Sci. USA 96: 7220-25, 1999) by homologous recombination. The vectors are similar to those traditionally used as targeting vectors with respect to the 5′ and 3′ homology arms and positive/negative selection schemes. However, the vectors also include sequence that, after recombination, either substitutes the human α2/δ1 coding sequence for the endogenous sequence, or effects base pair changes, exon substitutions, or codon substitutions that modify the endogenous sequence to encode the human α2/δ1. Once homologous recombinants have been identified, it is possible to excise any selection-based sequences (e.g., neo) by using Cre or Flp-mediated site directed recombination (Dymecki, Proc. Natl. Acad. Sci. 93: 6191-96, 1996). When substituting the human α2/δ1 sequence for the endogenous sequence, it is preferred that these changes are introduced directly downstream of the endogenous translation start site. This positioning preserves the endogenous temporal and spatial expression patterns of the α2/δ1 gene. The human sequence can be the full length human cDNA sequence with a polyA tail attached at the 3′ end for proper processing or the whole genomic sequence (Shiao et al., Transgenic Res. 8: 295-302, 1999). Further guidance regarding these methods of genetically modifying cells and non-human mammals to replace expression of an endogenous gene with its human counterpart is found, for example, in Sullivan et al., J. Biol. Chem. 272: 17972-80, 1997, Reaume et al., J. Biol. Chem. 271: 23380-88, 1996, and Scott et al., U.S. Pat. No. 5,777,194).

Another method for creating such “humanized” organisms is a two step process involving the disruption of the endogenous gene followed by the introduction of a transgene encoding the human sequence by pronuclear microinjection into the knock-out embryos. α2/δ1 function and therapeutic relevance can be further elucidated by investigating the phenotype of α2/δ1−/− non-human mammals and animals cells of the invention. For example, the genetically-modified α2/δ1−/− non-human mammals and animal cells can be used to determine whether the α2/δ1 plays a role in causing or preventing symptoms or phenotypes to develop in certain models of disease, e.g., central nervous system disorders including anxiety, depression, schizophrenia, bipolar disease, and the like; pain including neuropathic pain, and the like; and cardiovascular diseases including arrhythmia, hypertension, and the like. If a symptom or phenotype is different in a non-human mammal or animal cell as compared to a wildtype α2/δ1+/+) or heterozygote α2/δ1+/-) non-human mammal or animal cell, then the α2/δ1 polypeptide plays a role in regulating functions associated with the symptom or phenotype. Examples of testing that can be used to assess α2/δ1 function include comparing α2/δ1−/− mice to wildtype mice in terms of gabapentin and other α2/δ ligand binding; Tail Suspension Test (TST) for assessing sedation; Formalin Foot-Pad procedure for assessing susceptibility to formalin induced hyperalgesia, chronic constitutive injury (CCI)-induced static allodynia model of neuropathic pain, the Vogel Procedure for assessing anxiety; and Maximal Electro-Shock Procedure for assessing anticonvulsant activity. See Hain H S, Kinsora J J, Baron S P, Meltzer L T (2000) Society for Neuroscience Annual Meeting Abs. 336.12; Lotarski S M, Kinsora J J, Taylor C P, Baron S P (2002) Society for Neuroscience Annual Meeting Abs. 396.8; Krall et al. (1978) Epilepsia, August; 19(4):409-28.

In addition, under circumstances in which an agent has been identified as an α2/δ1 agonist or antagonist (e.g., the agent significantly modifies one or more of the α2/δ1 polypeptide activities when the agent is administered to a α2/δ1+/+ or α2/δ1+/− non-human mammal or animal cell), the genetically-modified α2/δ1−/− non-human mammals and animal cells of the invention are useful to characterize any other effects caused by the agent besides those known to result from the agonism or antagonism of α2/δ1 (i.e., the non-human mammals and animal cells can be used as negative controls). For example, if the administration of the agent causes an effect in a α2/δ1+/+ non-human mammal or animal cell that is not known to be associated with α2/δ1 polypeptide activity, then one can determine whether the agent exerts this effect solely or primarily through modulation of α2/δ1 by administering the agent to a corresponding α2/δ1−/− non-human mammal or animal cell. If this effect is absent, or is significantly reduced, in the α2/δ1−/− non-human mammal or animal cell, then the effect is mediated, at least in part, by α2/δ1. However, if the α2/δ1−/− non-human mammal or animal cell exhibits the effect to a degree comparable to the α2/δ1+/+ or α2/δ1+/− non-human mammal or animal cell, then the effect is mediated by a pathway that does not involve α2/δ1 signaling.

Furthermore, if an agent is suspected of possibly exerting an effect predominantly via a α2/δ1 pathway, then the α2/δ1−/− non-human mammals and animal cells are useful as negative controls to test this hypothesis. If the agent is indeed acting through α2/δ1, then the α2/δ1−/− non-human mammals and animal cells, upon administration of the agent, should not demonstrate the same effect observed in the α2/δ1+/+ non-human mammals or animal cells.

The genetically modified non-human mammals and animal cells of the invention can also be used to identify genes whose expression is differentially regulated in α2/δ1+/− or α2δ1−/− non-human mammals or animal cells relative to their respective wild-type control. Techniques known to those of skill in the art can be used to identify such genes based upon the present description. For example, DNA arrays can be used to identify genes whose expression is differentially regulated in α2/δ1+/− or α2/δ1−/− mice to compensate for a deficiency in α2/δ1 expression. DNA arrays are known to those of skill in the art (see, e.g., Aigner et al., Arthritis and Rheumatism 44: 2777-89, 2001; U.S. Pat. No. 5,965,352; Schena et al., Science 270: 467-470, 1995; DeRisi et al., Nature Genetics 14: 457-460, 1996; Shalon et al., Genome Res. 6: 639-645, 1996; and Schena et al., Proc. Natl. Acad. Sci. (USA) 93: 10539-11286, 1995).

In addition, the genetically modified non-human mammals and animal cells of the invention can also be used to identify proteins whose expression profile or postranslational modification is altered in α2/δ1+/− or α2/δ1−/− non-human mammals or animal cells relative to their respective wild-type control. Techniques known to those of skill in the art can be used to identify such proteins based upon the present description. For example, proteomic assays can be used to identify proteins whose expression profile or postranslational modification is altered in α2/δ1+/− or α2/δ14−/− mice to compensate for a deficiency in α2/δ1 expression. Proteomic assays are known to those of skill in the art (see, e.g., Conrads et al., Biochem. Biophys. Res. Commun. 290: 896-890, 2002; Dongre et al., Bioploymers 60: 206-211, 2001; Van Eyk Curr Opin Mol Ther 3: 546-553, 2001; Cole et al., Electrophoresis 21: 1772-1781, 2000; Araki et al., Electrophoresis 21: 180-1889, 2000).

The following examples are illustrative of the instant invention; they are not intended to limit the scope.

### EXAMPLES

### Example 1.

### Targeting Vector Construction

To generate a mouse that ubiquitously expresses an α2/δ1 subunit with a mutation at residue 217 (corresponding to residue 241 prior to cleavage of leader sequence) of the mouse α2/δ1 protein, genomic libraries were screened and replacement type targeting vectors were constructed using techniques essentially as described in Wattler S, Kelly M, and Nehls M (1999) BioTechniques 26:1150-1160, and further explained below.

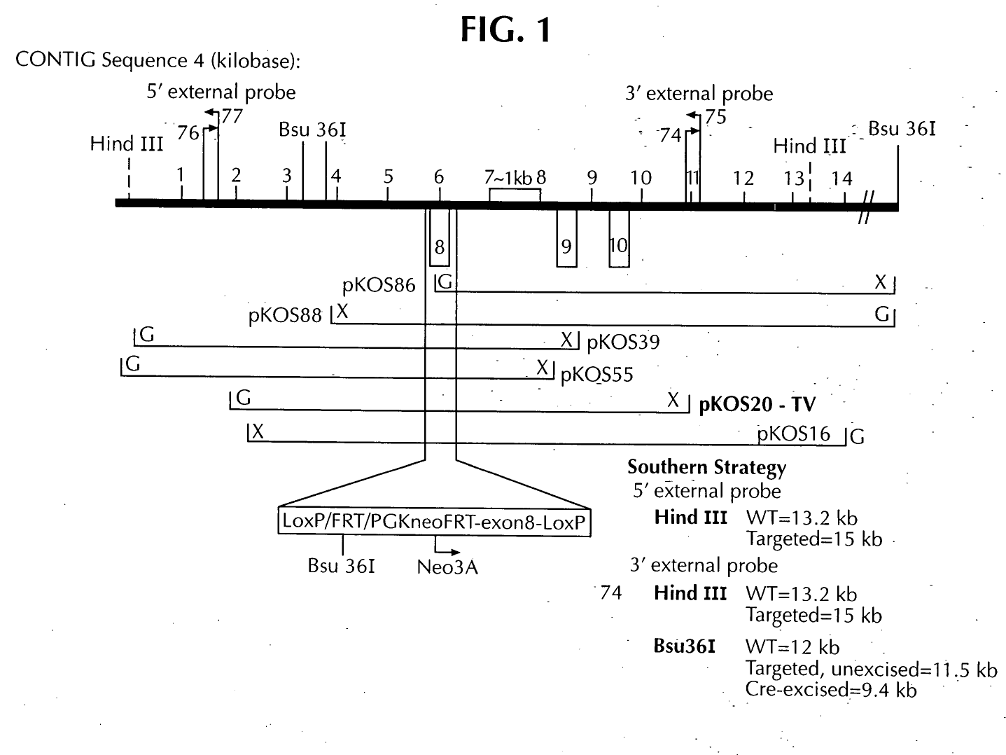

Sequences set forth in SEQ ID NO's: 1 and 2 were used in a PCR reaction using mouse genomic DNA as a template to generate a PCR fragment that contains genomic sequence from the α2/δ1 locus. The sequence of that PCR fragment is shown in SEQ ID NO: 3. This fragment was ligated to a yeast selection cassette that contains a nutritional marker for uracil (SEQ ID NO: 10). The ligation product containing the mouse genomic DNA and the yeast marker was transformed into a strain of yeast carrying a mouse genomic library. Homologous recombination occurs between the PCR fragment and genomic sequences carried in the library. This allows the isolation of genomic clones that are homologous to the α2/δ1 locus. Several clones were isolated and sequenced, and a contig was generated for the α2/δ1 locus containing exon 8. This contig is presented in SEQ ID NO: 4. This sequence contains ˜14kb of genomic DNA containing a portion of the α2/δ1 genomic DNA. One of the clones isolated (pKOS20) contained a portion of mouse genomic sequence containing exon 8. This genomic sequence is presented in SEQ ID NO: 5. A neo cassette, loxP sites and FRT sites were inserted into this sequence in positions flanking exon 8. See FIG. 1.

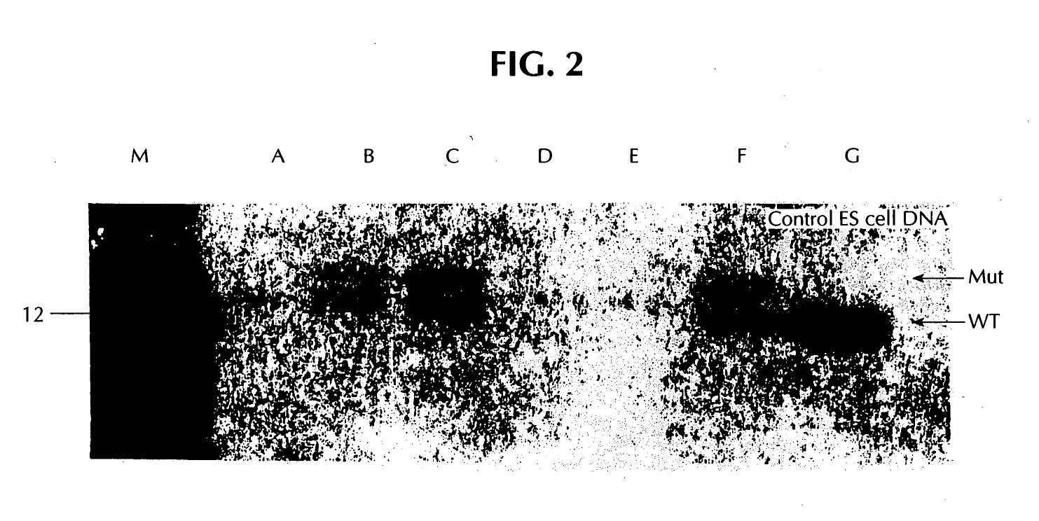

FIG. 1 depicts the organization of genomic clones in the α2/δ1 genomic region (76=SEQ ID NO: 15; 77=SEQ ID NO: 16; 74=SEQ ID NO: 13; 75=SEQ ID NO: 14; Neo3A=SEQ ID NO: 11. PKOS86, pKOS88, pKOS39, pKOS55, pKOS20-TV, pKOS 16 refer to genomic clones covering the exon 8 region of the α2/δ1 locus. The sequences set forth in these SEQ ID NO:'s were assembled in SEQ ID NO: 4, in order to generate the contig covering 14 kb of genomic α2/δ1 locus shown as top band, FIG. 1. PKOS20-TV, which refers to the targeting vector containing genomic SEQ ID NO: 5 was mutagenized to generate a targeting vector in which the codon for the arginine at position 217 (the c-terminal arginine in the only RRR motif in the polypeptide) is converted to an alanine using similar techniques to those described above. A PCR reaction was performed utilizing an oligonucleotide containing a modification of exon 8 which coded for this change. This mutagenic primer is shown in SEQ ID NO: 7. The wild-type sequence for exon 8 is shown in SEQ ID NO: 6. The mutagenic primer was used for two PCR reactions. One on the 5′ end, a PCR reaction was performed utilizing the primers set forth in SEQ ID NO: 7 and 8. On the 3′ end, a PCR reaction was performed utilizing the primers set forth in SEQ ID NO: 7 and 9. The products of these two PCR reactions were ligated onto the yeast selection cassette (SEQ ID NO: 10) and transformed into yeast carrying the targeting vector. The sequences recombined in the yeast and generated a sequence identical SEQ ID NO: 6 except with the residues coding for arginine 217 (AGA) converted to an alanine codon (GCA). This sequence comprising the residues encoding alanine 217 is set forth in SEQ ID NO: 20. This targeting vector was electroporated into ES cells. The targeting vector contains a marker for neomycin resistance, so cells that had been transfected were selected by growth in G418. ES cells that were resistant to G418 were isolated, and DNA isolated from the cells. The DNA was then subjected to PCR analysis using the primers in SEQ ID NO: 11 and 12. ES cells that are positive for the integration of the DNA were tested for the presence of a band at 900 base pairs in this PCR reaction. To confirm that the targeting vector had undergone homologous recombination and inserted in the α2/δ1 locus, DNA from positive ES cells was subjected to Southern analysis. Probes were generated from mouse genomic DNA using the primer pairs SEQ ID NO: 13- SEQ ID NO: 14 and SEQ ID NO: 15-SEQ ID NO: 16. These generate probes that are in regions flanking the integrated DNA. FIG. 2 presents a Southern blot using the 15-16 probe. A targeted ES cell in which homologous recombination has occurred would have a band at 15 KB which hybridizes to the probe. In contrast, the wildtype would have a band at 13.2 KB.

ES cells that have undergone homologous recombination were implanted into blastocysts derived from C57Bl/6 mice. These modified blastocysts were then implanted into pseudopregnant C57Bl/6 mothers. Chimeric pups were born. These chimeric pups were bred to C57Bl/6 mice and the offspring were then genotyped using the PCR approach outlined above. Animals in which germline transmission occurred were positive in this PCR assay.

Animals in which germline transmission had occurred were bred to generate congenic strains and to generate mice that could be used for testing.

### Example 2.

### Phenotypic Characterization of α2/δ1 Knockin Mice

2(A) Gabapentin Binding:

Membrane preparation and gabapentin binding were done essentially as described in Wang, M; Offord, J; Oxender D L; and SU T-Z (1999), Biochem. J. 342: 313-320. Membranes were prepared from whole mouse brains. These membranes were then used for binding of ³H gabapentin. Tracer levels of ³H gabapentin were used in binding. Total binding was determined in the absence of competitor. Non-specific binding was determined in the presence of 10 μM unlabeled pregabalin. FIG. 2 depicts a southern blot analysis of total genomic DNA isolated from ES cells and digested with Hind III ( M=Marker DNA; A=Es cell A; B=ES cell B; C=ES cell C; D=Es cell D; E=Es cell E; F=Es cell F; G=Control, untargeted ES cell DNA digested with HindIII). DNA was digested with HindIII, separated on an agarose gel then transferred to nitrocellulose. The nitrocellulose filter was probed with a 3′ probe consisting of the PCR fragment generated from genomic DNA using the primer pair SEQ ID NO: 13 and SEQ ID NO: 14.

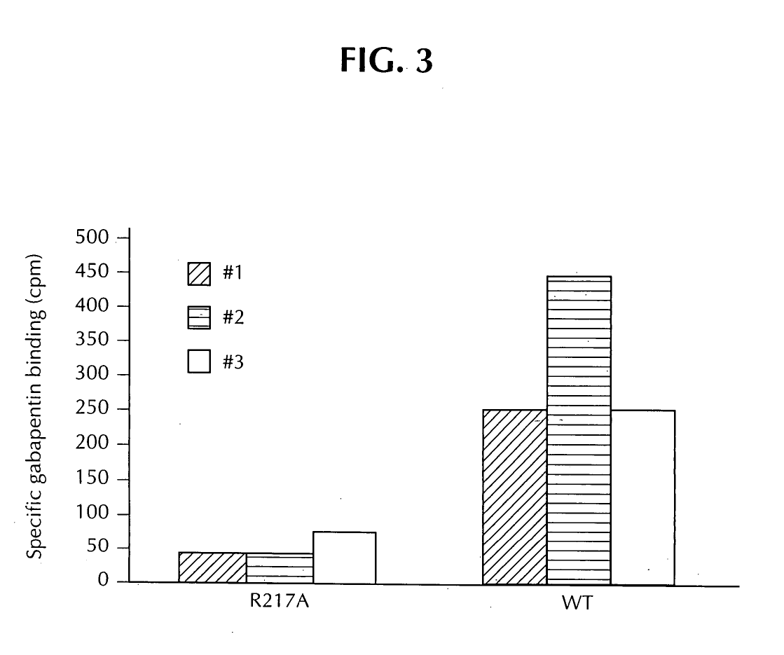

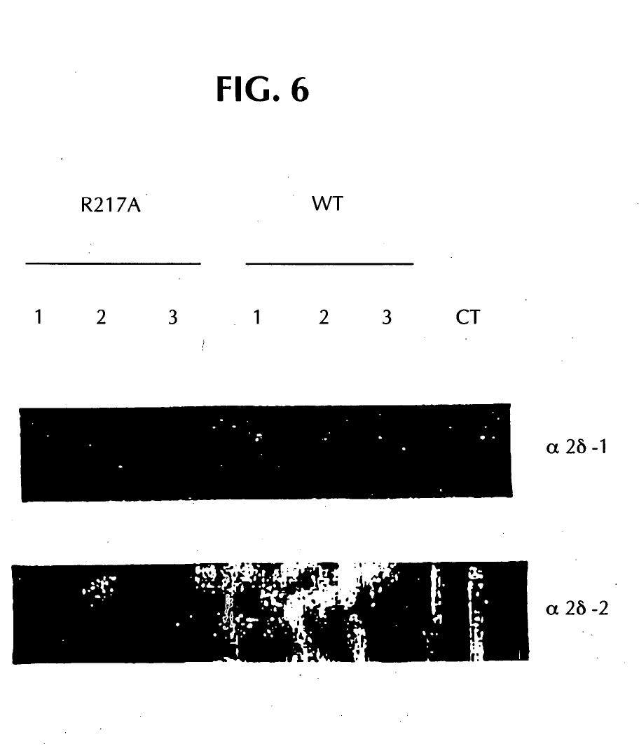

The results, set forth in FIG. 3, indicate that gabapentin binding to brain membranes from the R217A mutant mouse is reduced, or greatly abolished, in comparison to that from wildtype mice. Western blots indicated that levels of α2/δ1 and α2/δ2 polypeptides are not significantly changed as shown in FIG. 6. To generate the Western blots, membrane fractions were prepared from mouse brains. 10μg protein was loaded per well of polyacrylamide gels, subjected to electrophoresis, and blotting onto PVDF membranes. The blots were probed with an antibody specific for either α2δ1 or (α2δ2. In this manner, brain membranes from three different wildtype and three different mutant animals were probed.

2(B): Formalin-induced hyperalgesia in R217A mutant homozygous mice in comparison to that of heterozygous and wildtype mice/Formalin Foot-pad Procedure:

Male α2/δ1 R217A wild type, heterozygous, and homozygous mice, weighing 19-40 grams, were used for testing. Animals were maintained on a 12 hour light/dark cycle with food and water available ad libitum. Animals were food deprived overnight the day before testing. On test day, animals were acclimated to the observation chambers for approximately 60 min. The observation chambers were eight inch square boxes consisting of solid, acrylic plastic mirrors for the floor and 3 walls. The fourth wall was clear acrylic that allowed for observation. Animals were dosed PO with either vehicle or pregabalin, 120 minutes prior to testing at a dose volume 10 ml/kg. At test time, animals received a 25 μL injection of a 5% formalin solution in water in the plantar surface of the left hind foot. Immediately after the formalin injection, animals were observed for licking or biting of the injected foot over a 45-min period. These behaviors were timed using a hand-held stopwatch and recorded at 5 min intervals. Data was analyzed by the two distinct phases of responding. Early phase responding included Minutes 0-10, and late phase responding included Minutes 11-45. The total time spent licking by drug-treated animals in each phase was compared to the total time spent licking by vehicle-treated animals.

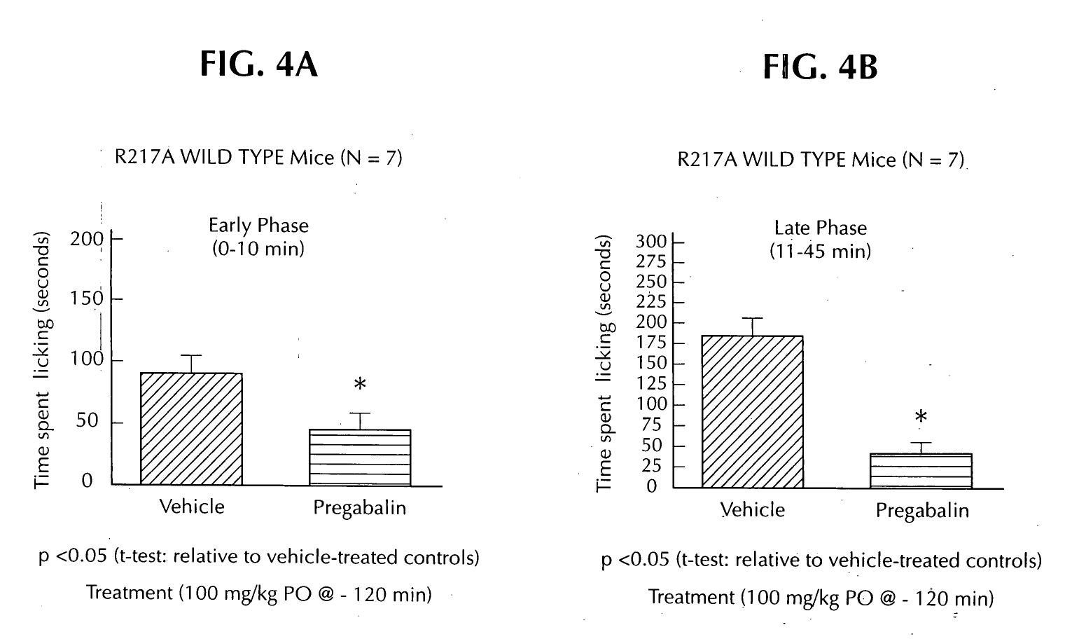

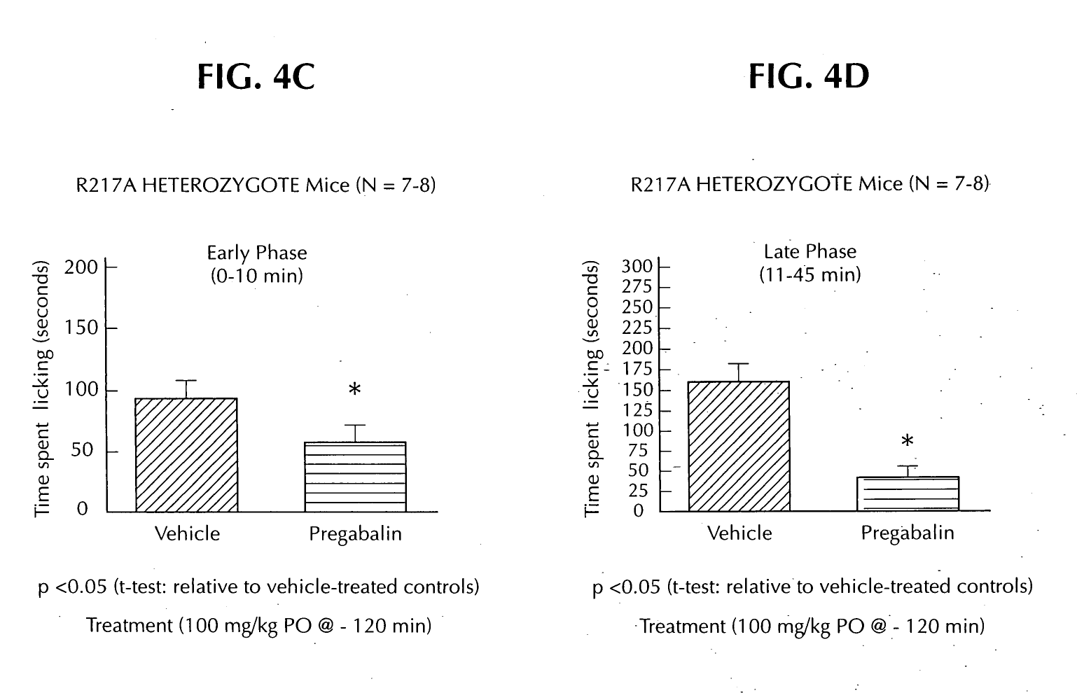

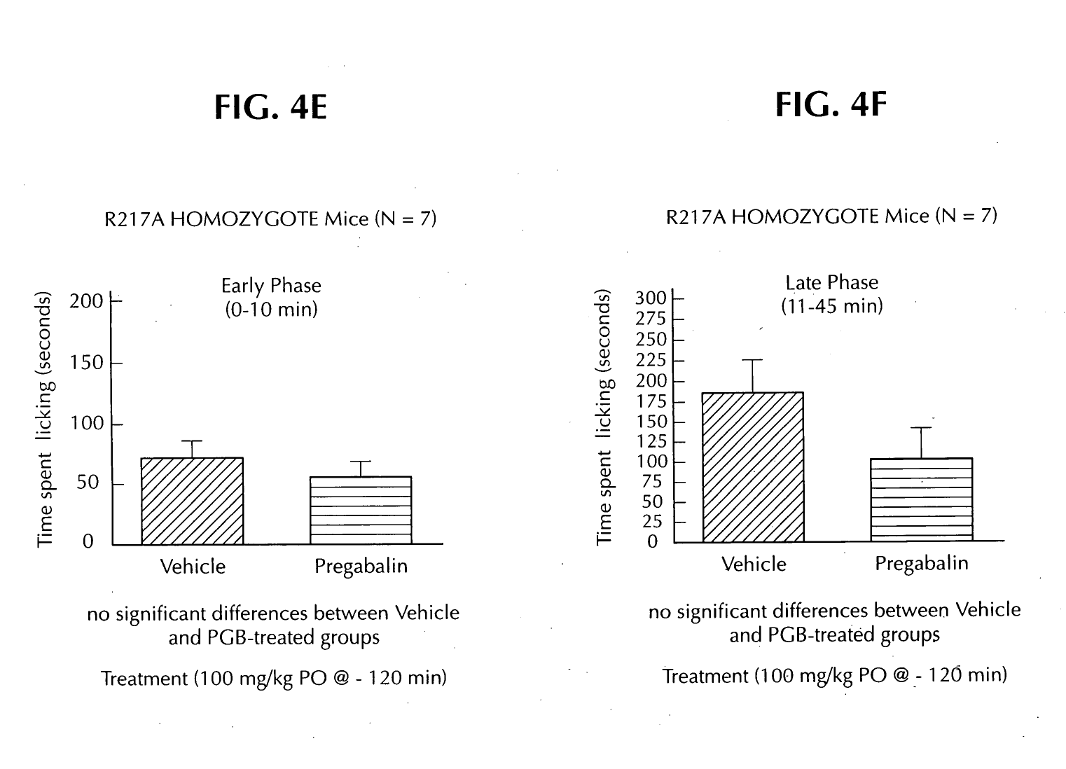

FIG. 4 depicts formalin-induced hyperalgesia in R217A mutant homozygous mice in comparison to that of heterozygous and wildtype mice. The results indicate that the mice in which the arg at position 217 was changed to an alanine show a reduced efficacy of pregabalin in this assay.

2(C) Effects of pregabalin in the Tail Suspension Test (TST) in R217A mutant and wildtype mice:

Male α2/δ1 R217A wild type and homozygous mice, weighing 22-35 grams, were used for testing. Animals were maintained on a 12 hour light/dark cycle with food and water available ad libitum. Animals were food deprived overnight the day before testing. On test day, animals were placed by groups of 8 into holding cages to acclimate for sixty minutes prior to dosing. Vehicle or pregabalin was administered 120 minutes prior to testing at a dose volume of 10 mL/kg. At test time, a piece of transparent tape was placed around the distal portion of the tail. The tape was then attached to a hook connected to an electro-mechanical apparatus that recorded the amount of time a mouse moved and the force used during movement. The hook was located at the top of a 24×18 cm testing chamber. Animals were completely suspended in air approximately 5 cm off the floor of the chamber for six minutes. Mean time of struggling and force of movement were then calculated for each group of eight.

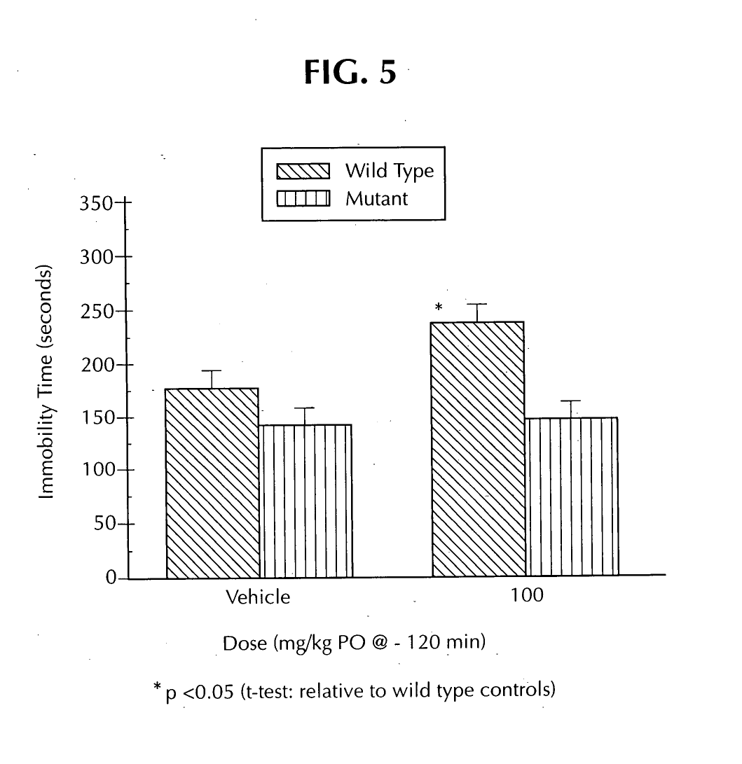

Animals are placed by groups of eight into holding cages to acclimate for thirty to sixty minutes prior to dosing. Treatments are generally administered 60 to 120 minutes prior to testing, although this also varies with specific compounds. At test time, a piece of scotch tape is placed around the distal portion of the tail. The tape is then attached to a hook that is connected to an electromechanical apparatus that records the amount of time a mouse moves and the force used during movement. The hook is located at the top of a 24×18 cm testing chamber. All animals are completely suspended in air approximately 5 cm off the floor of the chamber for six minutes. The apparatus automatically stops recording at the end of this time and animals are removed from the hook. FIG. 5 depicts the effects of pregabalin in the Tail Suspension Test (TST) in R217A mutant and wildtype mice.

The results indicate that pregabalin exerts its sedative and/or anxiolytic effect in TST through the α2/δ1 polypeptide. The results further indicate that this analgesic effect of pregabalin is mediated, at least partially, through the C-terminal flanking Arginine of RRR (R217) of wildtype mouse α2/δ1.

2(D) Allodynia

Chronic Constriction Injury (CCI) Model:

Animals were placed in an anaesthetic chamber and anaesthetised with a 2% isofluorane O₂ mixture. The right hind thigh is shaved and swabbed with 1% iodine. Animals were then transferred to a homeothermic blanket for the duration of the procedure and anaesthesia maintained during surgery via a nose cone. The skin was cut along the line of the thigh bone. The common sciatic nerve was exposed at the middle of the thigh by blunt dissection through biceps femoris. Proximal to the sciatic trifurcation, about 5mm of nerve was freed by inserting forceps under the nerve and the nerve gently lifted out of the thigh. The forceps were gently opened and closed several times to aid clearance of the fascia from the nerve. Suture was pulled under the nerve using forceps and tied in a simple knot until slight resistance was felt and then double knotted. The procedure was repeated until 3 ligatures (4-0 silk) were tied loosely around the nerve with approx 1 mm spacing. The incision was closed in layers and the wound treated with topical antibiotics.

Measurement of static allodynia

Animals were habituated to wire test cages prior to the assessment of allodynia. Static allodynia was evaluated by application of von Frey hairs (Stoelting, Wood Dale, Ill., U.S.A.) in ascending order of force (0.008, 0.02, 0.04, 0.07, 0.016, 0.4, 0.6, 1.0, 1.5 and 2.0 g) to the plantar surface of hind paws. Each von Frey hair was applied to the paw for a maximum of 6 seconds, or until a withdrawal response occurred. Once a withdrawal response to a von Frey hair was established, the paw was re-tested, starting with the filament below the one that produced a withdrawal, and subsequently with the remaining filaments in descending force sequence until no withdrawal occurred. Each animal had both hind paws tested in this manner. The lowest amount of force required to elicit a response was recorded as paw withdrawal threshold (PWT) in grams. Static allodynia was defined as present if animals responded to a stimulus of, or less than, 0.04 g, which is innocuous in normal mice. The von Frey hairs used in the present study give applied force in a logarithmic scale. Thus the data obtained in the current study was represented as medians and quartiles (representing the range of values) using a log scale.

Development of chronic constriction injury (CCI)-induced punctate allodynia in R217A and wild-type mice:

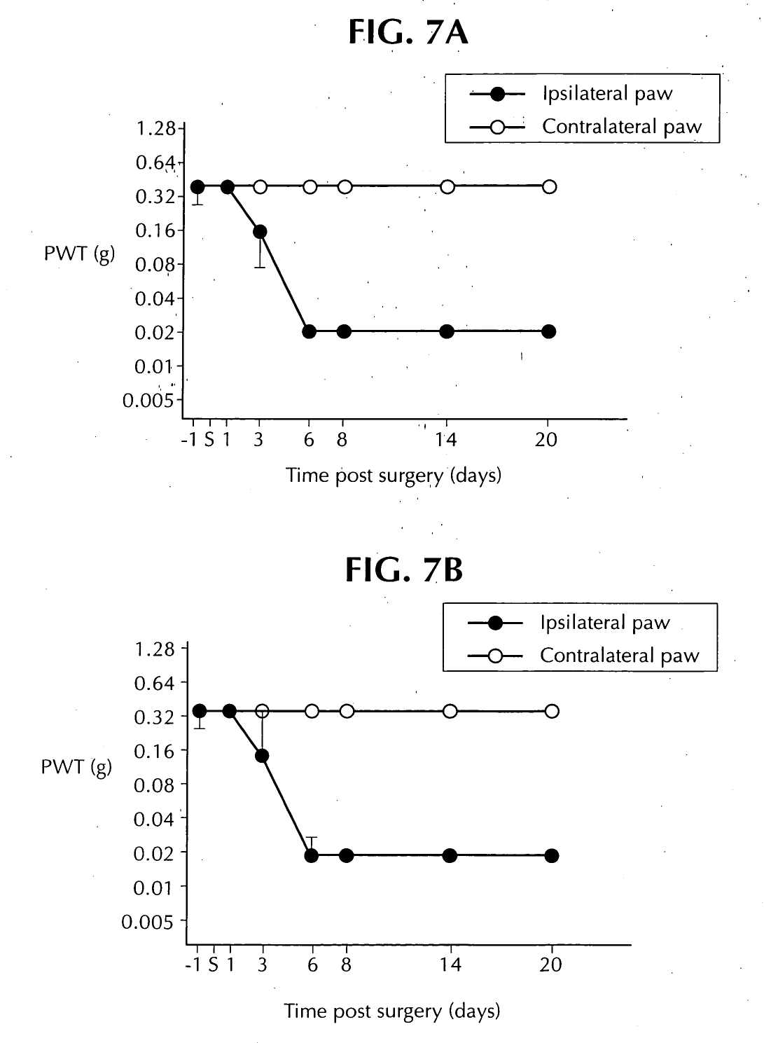

A model of nerve injury was used to further characterize the pain phenotype of the R217A and wild type mice. The CCI model was used for these studies as it is a well characterized model of neuropathic pain. Prior to CCI injury both knock-in and wild-type animals displayed similar baseline responses to punctate stimuli as measured using von Frey hairs. Following CCI injury an increased hypersensitivity developed in both groups of mice to the application of the von frey hairs which reached a peak seven days post-injury and was maintained for at least three weeks. A similar onset of pain behaviour was seen in both the knock-in and wild type mice. The results are depicted in FIG. 7.

Baseline (BL) paw withdrawal thresholds (PWT) to von Frey hairs were assessed.

Following drug administration PWT were re-assessed for up to 4 h. The static allodynia data is expressed as median force (g) required to induce a paw withdrawal in 6-7 animals per group (vertical bars represent 1^{st} and 3^{rd} quartiles) *P<0.05, **P<0.01***P<0.005 (Mann Whitney U test) from vehicle treated group at each time point.

Effect of pregabalin on CCI-induced static allodynia in the knock-in and wild-type mice:

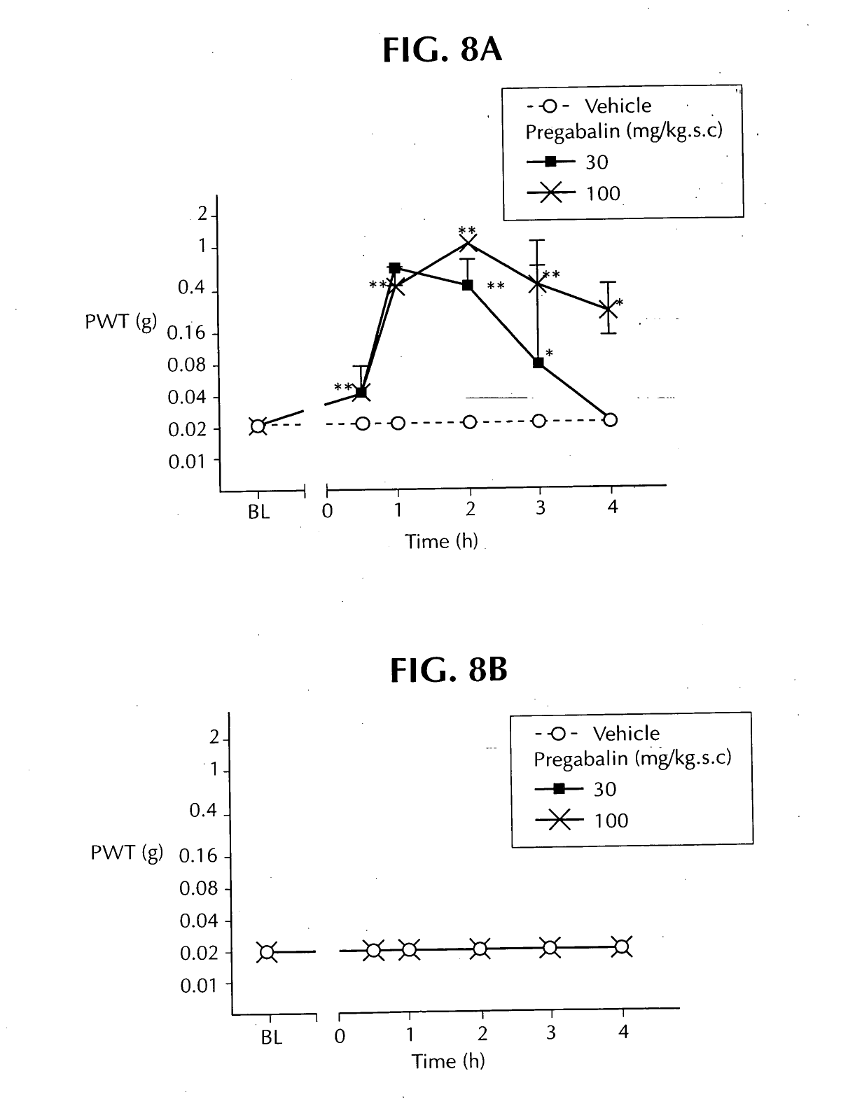

It was examined whether pregabalin produced an anti-allodynic action in both the knock-in and wild-type mice. In this study both knock-in and wild-type mice displayed a typical pain response to the application of a punctate stimulus following CCI surgery. The sub cutaneous administration of pregabalin (30-100mg/kg), produced a significant blockade of the CCI-induced punctate allodynia in the wild type mice. However, similar treatment of pregabalin produced no effect in the knock-in mice with pain behaviour analogous to vehicle treated controls. The results are depicted in FIG. 8. Baseline (BL) paw withdrawal thresholds (PWT) to von Frey hairs were assessed. Following drug administration PWT were re-assessed for up to 4 h. The static allodynia data is expressed as median force (g) required to induce a paw withdrawal in 6-7 animals per group (vertical bars represent 1^{st} and 3^{rd} quartiles) *P<0.05, **P<0.01***P<0.005 (Mann Whitney U test) from vehicle treated group at each time point.

The knock-in R217A and wild type mice display similar pain phenotype in chronic pain following the chronic constriction induced nerve injury. Pregabalin (30-100mg/kg, p.o.) displayed typical efficacy in CCI-induced allodynia in the wild type mice however it failed to demonstrate any efficacy following similar administration in the knock-in animals. These data demonstrate that the analgesic action of pregabalin are mediated by an interaction with the α2-δ-1 subunit of voltage gated calcium channels.

2(E) Role of (α2δ-1 subunit of calcium channels in the anxiolytic-like and anticonvulsant effects of pregabalin in mice: Use of the R217A point mutation in genetically modified mice.

The studies reported herein were carried out to elucidate the mechanism of action (MOA) of pregabalin by examining the role of α28δ-1 calcium channel auxiliary protein using the R217A point mutation in genetically modified mice. The Vogel procedure was used in assessing the anxiolytic effect of pregabalin while the Maximal electroshock procedure (MES) was used in assessing the anticonvulsent effect of pregabalin. The R217-A knock-in mice were made according to methods described above using the 2 different strains C57BL/6J, 129S6/SvEV. Data was also generated with the corresponding three different background strains as a comparison. Additionally, three different lines of mice were maintained denoted as: −/− (no mutation); +/− (heterozygote for the R217A mutation; and +/+ (homozygous for the R217 A mutation;).

In knock-in mice homozygous for the R217 A mutation, the anxiolytic and anticonvulsant effects are greatly reduced. In mouse Vogel water-lick conflict assay pregabalin does not produce a statistically significant increase in punished licking as compared to wild-type, heterozygous, or the back-ground strains. In the mouse anticonvulsant assay, pregabalin was less potent in protecting against seizures in the mutant mice than in the wild-type, heterozygous, or the back-ground strains. These data support a significant role of α28δ-1 binding in the mechanism of action of pregabalin, and in the clinically relevant behaviors affected by pregabalin. Theses data and the relevant methods are described below:

VOGEL Water-Lick Conflict

ANIMALS: Male mice, wild type (WT), heterozygous for the R217-A mutation (HET) and knockin (KI, heterozygous for the R217-A mutation) mice were received at six to 8 weeks old and housed 2 weeks prior to testing. Thirty-two WT, HET and KI mice were available from each shipment for testing, which resulted in pooling experiments when necessary. Two strains used to generate the mutant mice, C57BU6J (Jackson Laboratories), and 129S6/SvEV (Taconic Farms), were also evaluated. Albino or C57BL/6J-Tyr were also included in the experiments for comparison purposes. Male C57BL/6J (Jackson Laboratories), and 129S6/SvEV (Taconic Farms) mice were received at 8 weeks old in quantities sufficient for each study to be completed in one experiment. Parent strains were housed one week prior to testing. All animals were housed in a temperature and humidity controlled room under a 12L: 12D schedule (lights on @06:00 h). All mice were housed in isolators (3-5/cage) with the exception of C57BU6J mice that were housed on wire racks (12/cage).

VOGEL APPARATUS: The test apparatus consisted of 12 modular operant chambers (Coulbourn Instruments) measuring 7 W×7 L×12 H inches. On the side of each chamber (1.5 in. above the floor) a module optical lickometer was mounted. The lickometer was used to measure licking from a drink tube attached to a water bottle mounted on the exterior of the chamber. A photo beam was piped via glass rods across a gap at the end of the drink tube. The subject's tongue breaks the beam when it drinks from the tube and each lick was automatically recorded. Additionally, each standard test chamber was modified with an internal chamber made of clear Plexiglas measuring 3.5 D×6.75 W×1.5 H inches. The reduced chamber space limited the animal's activity and directed behavior towards the drink tube during a 10-minute session. The front and back of the test chamber was made of clear Plexiglas. The front door was covered to reduce distractions from inside the test room. The back of the test chamber faced a wall, away from the flow of traffic in the testing room, and remained uncovered to provide the opportunity for observations. Shock was delivered between the grid floor and the drink tube using a (Coulbourn) programmable universal shocker for a duration of one second, but terminated immediately when contact between the animal and drink tube was broken. For each strain and mutant genotype, shock intensities were experimentally determined as the intensity that consistently suppressed drinking greater than 80% when compared to a concurrent no-shock control group.

PROCEDURE: On day one, after 24 hours of water deprivation, experimental subjects were placed in the test chambers and allowed to drink unpunished for a 10-minute training session. Mice were required to complete a 100-150 lick criteria during the training session. Subjects that completed the training criteria prior to the end of the 10-minute session were removed so that drinking was restricted in all subjects to 25% of their total daily intake. Mice that failed to emit 100-150 licks were eliminated from the study. Immediately after the day one session mice were returned to their home cages and deprived of water and food for an additional 24 hours. On test day two, mice were dosed by intraperitoneal (IP) injection with vehicle or test compound. After a 60 or 120-minute absorption period mice were placed into the test chambers for a 10-minute test session. During the test, shock was delivered on a fixed ratio (FR) 10 schedule. Therefore, after every 10^{th} lick the subject received a mild shock. Hence, a conflict situation exists; mice were motivated to drink but responding was inhibited by the shock. A low number of shock episodes accepted reflects anxiety-related behavior. Standard anxiolytic drugs produce effects that allow mice to overcome the behavioral inhibition and drink despite the shock. Compounds that significantly increase the number of shock episodes over concurrently run controls were presumed to produce anxiolytic-like effects. All data were analyzed using a one-way ANOVA/Dunn's Method.

DRUGS: Pregabalin was dissolved in physiological saline for all experiments. The positive control diazepam was suspended in 2% cremophor in physiological saline. All drugs were dosed in a volume of 10 mL/kg.

Experimental Design

Diazepam and Pregabalin Testing in Parent Strains:

The shock intensity used for each strain was previously determined experimentally as the intensity that consistently suppressed drinking greater than 80% (data not shown). Similar suppression of 80% in each strain provided a consistent baseline for evaluating drug. Dose-response studies on diazepam and pregabalin were tested in C57BL/6J, C57BL/6J-Tyr and 129S6/SvEV mouse strains to determine the minimally effective dose (MED) for each drug in each strain. C57BL/6J mice were tested at 0.4 mA, C57BL/6J-Tyr mice were tested at 0.1 mA and 129S6/SvEV mice were tested at 0.6 mA. Each dose-response study included a no-shock vehicle control group directly compared to the concurrent vehicle punished-responding group to evaluate the suppression of drinking. Diazepam (−1 hr, IP for all strains) was tested in C57BL/6J mice at doses of 1, 3.2, 10 and 32 mg/kg and pregabalin (−2 hr, IP for all strains) was tested at doses of 3.2, 10, 32 and 100 mg/kg. Diazepam was tested in C57BL/6J-Tyr mice at doses of 0.32, 1 and 3.2 mg/kg and pregabalin was tested at doses of 3.2, 10, 32 and 100 mg/kg. Diazepam was tested in 129S6/SvEV mice at doses of 0.32, 1 and 3.2 mg/kg and pregabalin was tested at doses of 3.2, 10 and 32 mg/kg. For all experiments, sedation was noted when both symptoms of ataxia (unsteady gait) and a reduced response to handling were observed after dosing and described as mild, moderate or severe.

Diazepam and Pregabalin Testing in the Mutant Mice

The shock intensity used for each genotype was previously determined experimentally as the intensity that consistently suppressed drinking 80% or more (data not shown). Similar suppression of 80% in each genotype provided a consistent baseline for evaluating drug. Dose response studies on diazepam (1, 3.2 and 10 mg/kg, −1 hr, IP) and pregabalin (10, 32 and 100 mg/kg, −2 hr, IP) were tested in WT, HET and KI mice to determine the MED for each drug in each genotype. WT mice were tested at 0.1 mA, HET mice were tested at 0.4 mA and KI mice were tested at 0.7 InA. Each experiment included a no-shock vehicle control group for direct comparison to the concurrent vehicle punished-responding group to determine percent suppression of drinking. A single dose of diazepam (3.2 mg/kg) or pregabalin (100 mg/kg) was also selected based on non-significant increases noted in the dose response studies that may have been confounded by pooling multiple studies. To for further evaluate these increases and attempt to reduce variability associated with pooling multiple studies the single dose experiments were carried out in all three genotypes. Sedation was noted by the observer as previously described. MES: Anticonvulsant effectiveness was determined by using an electroconvulsive seizure model as described elsewhere [White et al., 1995]. Mice had free access to food and water and were housed in a temperature and light-controlled environment (12 h on/12 h off) prior to drug administration. Electroshock in mice was delivered using corneal electrodes with varied electroshock intensity. Current was delivered (60 Hz sinusoidal current, 11 to 17 mA baseline to peak vs. 50 mA for 0.2 s) using a constant-current stimulator (Wahlquist Instruments, Salt Lake City, Utah). The intensities of current necessary to produce tonic extensor seizures for each mouse strain and mutant lines in all of the mice were determined. These currents were then used for further testing of mice with pregabalin. All dose-response testing was conducted at the previously determined time of peak drug effect. Groups of 10 mice each were give various intraperitoneal doses of pregabalin dissolved in 0.9% saline, diazepam (veh) or phenytoin and given in a volume of 10 ml/kg of body weight. ED₅₀ values (calculated dose required to inhibit the hindlimb tonic-extensor component of the maximal electroshock seizure in 50% of the animals tested) were determined by probit analysis [Litchfield, Wilcoxon], and used to compare relative potency between compounds. At least four groups of mice and each animal was employed once. The animals, pretreated with different doses of the tested drugs, were challenged with one electroshock at previously determined times of peak drug effect (pregabalin: 120 min; diazepam: 60 min; phenytoin; 60 min)

Results/Vogel Procedure

R217A/Patent Strain Correlations

| Strain | mA > 80% | Pregabalin | Diazepam |
|---|---|---|---|
| Wild Type | 0.1 mA | 32 | 3.2 |
| Hets | 0.4 mA | 10 | 3.2 |
| Knockins | 0.7 mA | inactive | inactive |
| | | non-significant | |
| | | increase @ 100 | |
| 129S6/SvEv | 0.6 mA | 32 | inactive |
| C57 albino | 0.1 mA | 10 | 3.2 |
| C57BL/6J | 0.4 mA | 10 | 3.2 |

α2δ-1 binding is important for the anxiolytic-like effects of pregabalin
Pregabalin did not produce a significant anxiolytic-like effect in R217A knockins, and no evidence was obtained relating phenotype to particular parent strain
Diazepam (+ control) failed to produce an anxiolytic-like effect in R217A knockins. Data suggests 129S6-parent strain's genotype may be responsible for pharmacological profile of diazepam

Results/MES

The MES procedure results are set forth in FIG. 9. Further in regards to FIG. 9, all background strains as well as mutant, heterozygous mutant, and wildtype (all lines) were fully responsive to the anticonvulsant effects of phenytoin and diazepam. −/− mice were less sensitive to effects of pregabalin than the +/+ mice. Anticonvulsant effects of pregabalin were reduced in −/− mice as compared to the +/+ mice.

### Example 3

SEQUENCE DATA Features

SEQ ID NO. 17

is encoded by exon 8, and sets forth a mouse polypeptide having Arg to Ala mutation in comparison to wildtype; the mutation shown as residue 22.

SEQ ID NO. 18

is encoded by exons 8 and 9, and sets forth a mouse polypeptide having Arg to Ala mutation at exon 8 in comparison to wildtype; the mutation shown as residue 22.

SEQ ID NO. 19

is encoded by exons 8, 9, and 10, and sets forth a mouse polypeptide having Arg to Ala mutation at exon 8 in comparison to wildtype; the mutation shown as residue 22.

SEQ ID NO. 20

Sets forth a nucleotide sequence encoding the polypeptide set forth in SEQ ID No. 17.

SEQ ID NO. 21

Sets forth a mouse genomic DNA sequence comprising sequences encoding the polypeptide set forth in SEQ ID No. 18.

SEQ ID No. 22

Sets forth a nucleotide sequence encoding the polypeptides set forth in SEQ ID No. 18.

SEQ ID No. 23

Sets forth a mouse genomic DNA sequence comprising sequences encoding the polypeptide set forth in SEQ ID No. 19.

SEQ ID No. 24

Sets forth a nucleotide sequence encoding mouse the polypeptide sets forth in SEQ ID No. 19.

1. A genetically-modified, non-human mammal comprising an α2/δ1 gene comprising an R217-like mutation.

2. A genetically-modified, non-human mammal, wherein the modification results in a mutated α2/δ1 gene encoding a polypeptide selected from the group consisting of:
a) An α2/δ1 polypeptide comprising an arginine to non-arginine substitution in at least one of the two flanking arginines in an RRR motif unique to said polypeptide;
b) An α2/δ1 polypeptide comprising an arginine to aliphatic amino acid substitution in at least one of the two flanking arginines in an RRR motif unique to said polypeptide;
c) An α2/δ1 polypeptide comprising an arginine to alanine substitution in at least one of the two flanking arginines in an RRR motif unique to said polypeptide;
d) An α2/δ1 polypeptide comprising a deletion of at least one of the flanking arginines in an RRR motif unique to said polypeptide;
e) An α2/δ1 polypeptide comprising a deletion of up to 9 residues immediately N-terminal to an RRR motif unique to said polypeptide, a deletion of up to 5 residues immediately C-terminal to an RRR motif unique, and a deletion of at least one of the flanking arginines in an RRR motif unique to said polypeptide;
f) An α2/δ1 polypeptide comprising a deletion of up to of up to 9 residues immediately N-terminal to an RRR motif unique to said polypeptide, and a deletion of at least one of the flanking arginines in an RRR motif unique to said polypeptide;
g) An α2/δ1 polypeptide comprising a deletion of up to 5 residues immediately C-terminal to an RRR motif unique, and a deletion of at least one of the flanking arginines in an RRR motif unique to said polypeptide; and
h) An α2/δ1 polypeptide according to a)-g) having at least one conservative amino acid substitution at a position other than a flanking arginines in said RRR motif.

3. A genetically-modified, non-human mammal, wherein the modification results in a mutated α2/δ1 gene encoding a polypeptide selected from the group consisting of:
a) An α2/δ1 polypeptide that is identical to a wildtype α2/δ1 polypeptide except that it has an arginine to non-arginine substitution in at least one of the two flanking arginines in an RRR motif unique to said polypeptide;
b) An α2/δ1 polypeptide that is identical to a wildtype α2/δ1 polypeptide except that it has an arginine to aliphatic amino acid substitution in at least one of the two flanking arginines in an RRR motif unique to said polypeptide;
c) An α2/δ1 polypeptide that is identical to a wildtype α2/δ1 polypeptide except that it has an arginine to alanine substitution in at least one of the two flanking arginines in an RRR motif unique to said polypeptide;
d) An α2/δ1 polypeptide that is identical to a wildtype α2/δ1 polypeptide except that it has a deletion of at least one of the flanking arginines in an RRR motif unique to said polypeptide;
e) An α2/δ1 polypeptide that is identical to a wildtype α2/δ1 polypeptide except that it has a deletion of up to 9 residues immediately N-terminal to an RRR motif unique to said polypeptide, a deletion of up to 5 residues immediately C-terminal to an RRR motif unique, and a deletion of at least one of the flanking arginines in an RRR motif unique to said polypeptide;
f) An α2/δ1 polypeptide that is identical to a wildtype α2/δ1 polypeptide except that it has a deletion of up to of up to 9 residues immediately N-terminal to an RRR motif unique to said polypeptide, and a deletion of at least one of the flanking arginines in an RRR motif unique to said polypeptide;
g) An α2/δ1 polypeptide that is identical to a wildtype α2/δ1 polypeptide except that it has a deletion of up to 5 residues immediately C-terminal to an RRR motif unique, and a deletion of at least one of the flanking arginines in an RRR motif unique to said polypeptide; and
h) An α2/δ1 polypeptide according to a)-g) having at least one conservative amino acid substitution at a position other than a flanking arginine in said RRR motif;
wherein said α2/δ1 polypeptide lacks its leader sequence.

4. The genetically modified, non-human mammal of claim 3 wherein said wildtype α2/δ1 polypeptide is set forth in SEQ ID NO: 25, 26, 27, 28, 29, 30, or 31.

5. A genetically-modified, non-human mammal, wherein the modification results in a mutated α2/δ1 gene encoding a polypeptide selected from the group consisting of:
a) An α2/δ1 polypeptide that is identical to a wildtype α2/δ1 polypeptide except that it has an amino acid other than arginine at position 215, 217 or both;
b) An α2/δ1 polypeptide that is identical to a wildtype α2/δ1 polypeptide except that it has an aliphatic amino acid at position 215, 217 or both;
c) An α2/δ1 polypeptide that is identical to a wildtype α2/δ1 polypeptide except that it has an alanine at position 215, 217 or both;
d) An α2/δ1 polypeptide that is identical to a wildtype α2/δ1 polypeptide except that it has a lysine at position 215, 217 or both; and
e) The wildtype mammalian α2/δ1 polypeptide according to A)-d) having at least one conservative amino acid substitution at a position other than residue 215 and 217.

6. The mammal of claim 2, wherein said mammal exhibits at least one phenotypic characteristic selected from the group consisting of:
a) the phenotypic characteristic of reduced α2/δ ligand binding to central nervous system of said mammal;
b) the phenotypic characteristic of reduced gabapentin binding to central nervous system of said mammal;
c) the phenotypic characteristic of reduced analgesic efficacy of an α2/δ ligand in said mammal;
d) the phenotypic characteristic of reduced analgesic efficacy of pregabalin in said mammal;
e) the phenotypic characteristic of reduced sedative efficacy of an α2/δ ligand in said mammal wherein said mammal is subjected to a sedation test;
f) the phenotypic characteristic of reduced anticonvulsant efficacy of an α2/δ ligand in said mammal wherein said mammal is subjected to a sedation test; and
g) the phenotypic characteristic of reduced anxiolytic efficacy of an α2/δ ligand in said mammal wherein said mammal is subjected to a sedation test.

7. The mammal of claim 2, wherein said mammal is a rodent.

8. The rodent of claim 7, wherein said rodent is a mouse.

9. The non-human mammal of claim 2, wherein said mammal is homozygous for said modification.

10. An isolated nucleic acid molecule having a sequence encoding a polypeptide comprising the sequence selected from the group consisting of:
a) A polypeptide sequence set forth in SEQ ID NO: 17,
b) A polypeptide sequence set forth in SEQ ID NO: 18, and
c) A polypeptide sequence set forth in SEQ ID NO: 19.

11. The isolated nucleic acid molecule according to claim 10 a) comprising a nucleotide sequence set forth in SEQ ID NO: 20.

12. The isolated nucleic acid molecule according to claim 10 b) comprising a nucleotide sequence set forth in SEQ ID NO: 21 or SEQ ID NO: 22.

13. The isolated nucleic acid molecule according to claim 10 c) comprising a nucleotide sequence set forth in SEQ ID NO: 23 or SEQ ID NO: 24.

14. A genetically-modified, non-human mammal comprising the nucleic acid sequence of claim 10.

15. A targeting vector for producing a transgenic animal, said vector comprising a nucleic acid having a nucleotide sequence encoding a polypeptide according to claim 2.

16. A host cell comprising the vector of claim 15.

17. A genetically-modified animal cell, wherein the modification comprises a mutated gene encoding a polypeptide according to claim 2.

18. The animal cell of claim 17, wherein said cell is an embryonic stem (ES) cell or an ES-like cell.

19. The animal cell of claim 17, wherein said cell is isolated from a genetically-modified, non-human mammal containing a modification that results in a mutated gene.

20. The animal cell of claim 19, wherein said cell is an embryonic fibroblast, stem cell, neuron, skeletal or cardiac muscle cell, myoblast, brown or white adipocyte, hepatocyte, or pancreatic P cell.

21. The animal cell of claim 17, wherein said cell is murine.

22. The animal cell of claim 17, wherein said cell is human.

23. The animal cell of claim 17, wherein said cell is homozygous for said modification.

24. A method of identifying a gene that demonstrates modified expression as a result of reduced α2/δ1 activity in an animal cell, said method comprising assessing the expression profile of an animal cell containing a genetic modification that disrupts a cα2/δ1 gene, and comparing said profile to that from a wildtype cell.

25. The method of claim 24, wherein said cell is homozygous for a genetic modification that disrupts the α2/δ1 gene.

26. A method of identifying a protein that demonstrates modified expression or post-translational modification as a result of reduced α2/δ1 activity in an animal cell, said method comprising assessing the proteomic profile of an animal cell containing a genetic modification that disrupts a α2/δ1 gene, and comparing said profile to that from a wildtype cell.

27. A method of claim 26, wherein said cell is homozygous for a genetic modification that disrupts the α2/δ1 gene.

28. A method for producing a transgenic animal having a modified response in an α2/δ1-mediated disorder or activity relative to a wildtype animal, said method comprising:
a) transfecting ES cells with a targeting vector for producing a transgenic animal, said vector comprising a nucleic acid having a nucleotide sequence encoding a polypeptide according to claim 2;
b) selecting transfected cells undergone homologous recombination;
c) implanting said selected transfected cells into blastocysts;
d) producing transgenic animals from said blastocysts.

29. The method of claim 28, wherein said activity or disorder of claim 28 is selected from pain, hyperalgesia, anxiety, sedation, epilepsy, convulsion.

30. A method for determining whether the physiological effect of a compound on a disorder or activity involves α2/δ1 subunit polypeptide residues that mediate the physiological effect of an α2/δ ligand, said method comprising
a) providing a first group of mammals according to claim 2 and, a second group of corresponding wildtype mammals,
b) treating a first subset of each said group with an α2/δ ligand,
c) treating a second subset of each said group with a test compound,
d) testing each subset for an activity or disorder associated with α2/δ1, and
e) comparing the response of each said each said groups and subsets.

31. The method of claim 30 wherein said activity or disorder of is selected from pain, hyperalgesia, anxiety, sedation, epilepsy, convulsion.

32. The method of claim 30 wherein said α2/δ ligand is gabapentin.

33. The method of claim 30 wherein said α2/δ1 ligand is pregabalin.

34. A method for identifying compounds that exert their physiological effect on a disorder or activity through an α2/δ1 subunit polypeptide, said method comprising
a) providing a first group of mammals according to claim 2 and a second group of corresponding wildtype mammals,
b) treating each said group with a test compound,
c) testing each group for an activity or disorder associated with α2/δ1, and
d) comparing the response of each said each said groups.

35. A method for identifying compounds that exert their physiological effect on a disorder or activity through an α2/δ1 subunit polypeptide, said method comprising
a) providing a first group of mammals according to claim 2 and a second group of corresponding wildtype mammals,
b) treating a first subset of each said group with a ligand that binds an α2/δ1 subunit polypeptide,
c) treating a second subset of each said group with a test compound,
d) testing each subset for an activity or disorder associated with α2/δ1, and
e) comparing the response of each said each said groups and subsets.

36. The method of claim 35 wherein said ligand is gabapentin.

37. The method of claim 35 wherein said activity or disorder is selected from pain, hyperalgesia, anxiety, sedation, epilepsy, convulsion.

38. A method for determining a role of α2/δ1 polypeptide in an activity or disorder, said method comprising
a) providing a first group of mammals according to claim 2 and a second group of corresponding wildtype mammals,
b) subjecting each said group to a procedure indicative of an activity or disorder, and
c) comparing the response of each said group.

39. The method of claim 38 wherein said activity or disorder of is selected from pain, hyperalgesia, anxiety, sedation, epilepsy, convulsion.

40. The method of claim 38 wherein said procedure is selected from α2/δ ligand binding, gabapentin binding, formalin foot-pad procedure, Tail suspension test, Maximal electro-shock, and Vogel procedure.
